(19) United States
(12) Patent Application Publication
(10) Pub. No.: US 2019/0191707 A1
(43) Pub. Date: Jun. 27, 2019

(54) COMPOSITIONS COMPRISING BACILLUS LICHENIFORMIS AND BACILLUS SUBTILIS AND METHODS OF USE FOR CONTROLLING FUNGAL PATHOGENS

(71) Applicant: FMC Corporation, Philadelphia, PA (US)

(72) Inventors: Safiyh Taghavi, Chapel Hill, NC (US); Daniel Van Der Lelie, Chapel Hill, NC (US); Jaeheon Lee, Holte (DK); Roderick Mcleod, Lethbridge (CA); John E. Kibbee, Guelph (CA); Kevin R. Brost, Calgary (CA); Guozhi Wang, Oakland Gardens, NY (US); Vincent J. Spadafora, Yardley, PA (US); Matthew F. Pye, Newton, PA (US)

(21) Appl. No.: 15/563,101

(22) PCT Filed: Aug. 30, 2017
(86) PCT No.: PCT/US2017/049412

§ 371 Date: Sep. 29, 2017

Related U.S. Application Data
(60) Provisional application No. 62/381,860, filed on Aug. 31, 2016.
(60) Provisional application No. 62/381,814, filed on Aug. 31, 2016.

(51) Int. Cl. A01N 63/02; A01N 25/04; A01N 25/10; C12N 1/20; C12R 1/10; C12R 1/125

(52) U.S. Cl. CPC A01N 63/02; A01N 25/04; C12R 1/125; C12N 1/20; C12R 1/10; A01N 25/10

(57) ABSTRACT

Compositions and methods include a combination of a strain of Bacillus licheniformis CH200 and a strain of Bacillus subtilis CH201 for delivery to plants and plant seeds, the combination having activity against plant fungal pathogens. In particular, the combination of strains is useful for increasing yield in crops including soybean and corn in the presence of plant pathogens. The Bacillus licheniformis CH200 and Bacillus subtilis CH201 strains can be applied alone or in combination with other microbial, biological, or chemical insecticides, fungicides, nematicides, bacteriocides, herbicides, plant extracts, plant growth regulators, and fertilizers.

### CROSS REFERENCE TO RELATED APPLICATIONS

This application claims the benefit of U.S. provisional application No. 62/381,860, filed Aug. 31, 2016 and U.S. provisional application No. 62/381,814, filed Aug. 31, 2016, the disclosures of which are hereby incorporated herein by reference in their entireties.

### TECHNICAL FIELD

The presently disclosed subject matter relates to compositions comprising isolated microbial strains for application to plants, plant seeds, and the soil surrounding plants to benefit plant growth and to treat plant fungal disease. In certain cases the microbial strains are delivered to the plants, plant seeds, and the soil surrounding plants in combination with a chemical active agent having antimicrobial properties.

### BACKGROUND

A number of microorganisms having beneficial effects on plant growth and health are known to be present in the soil, to live in association with plants specifically in the root zone (Plant Growth Promoting Rhizobacteria “PGPR”), or to reside as endophytes within the plant. Their beneficial plant growth promoting properties include nitrogen fixation, iron chelation, phosphate solubilization, inhibition of non-beneficial microorganisms, resistance to pests, Induced Systemic Resistance (ISR), Systemic Acquired Resistance (SAR), decomposition of plant material in soil to increase useful soil organic matter, and synthesis of phytohormones such as indole-acetic acid (IAA), acetoin and 2,3-butanediol that stimulate plant growth, development and responses to environmental stresses such as drought. In addition, these microorganisms can interfere with a plant's ethylene stress response by breaking down the precursor molecule, 1-aminocyclopropane-1-carboxylate (ACC), thereby stimulating plant growth and slowing fruit ripening. These beneficial microorganisms can improve soil quality, plant growth, yield, and quality of crops. Various microorganisms exhibit biological activity such as to be useful to control plant diseases. Such biopesticides (living organisms and the compounds naturally produced by these organisms) are safer and more biodegradable than synthetic fertilizers and pesticides.

Fungal phytopathogens, including but not limited to Botrytis spp. (e.g. Botrytis cinerea), Fusarium spp. (e.g. F. oxysporum and F. graminearum), Rhizoctonia spp. (e.g. R. solani), Magnaporthe spp., Mycosphaerella spp., Puccunia spp. (e.g. P. recondita), Phytopthora spp. and Phakopsora spp. (e.g. P. pachyrhizi), are one type of plant pest that can cause severe economic losses in the agricultural and horticultural industries. Chemical agents can be used to control fungal phytopathogens, but the use of chemical agents suffers from disadvantages including high cost, lack of efficacy, emergence of resistant strains of the fungi, and undesirable environmental impacts. In addition, such chemical treatments tend to be indiscriminant and may adversely affect beneficial bacteria, fungi, and arthropods in addition to the plant pathogen at which the treatments are targeted. A second type of plant pest are bacterial pathogens, including but not limited to Erwinia spp. (such as Erwinia chrysanthemi), Pantoea spp. (such as P. citrea), Xanthomonas (e.g. Xanthomonos campestris), Pseudomonas spp. (such as P. syringae) and Ralstonia spp. (such as R. soleacearum) that cause severe economic losses in the agricultural and horticultural industries. Similar to pathogenic fungi, the use of chemical agents to treat these bacterial pathogens suffers from disadvantages. Viruses and virus-like organisms comprise a third type of plant disease-causing agent that is hard to control, but to which bacterial microorganisms can provide resistance in plants via. Induced systemic resistance (ISR). Thus, microorganisms that can be applied as biofertilizer and/or biopesticide to control pathogenic fungi, viruses, and bacteria are desirable and in high demand to improve agricultural sustainability. A final type of plant pathogen includes plant pathogenic nematodes and insects, which can cause severe damage and loss of plants.

Some members of the species Bacillus have been reported as blocontrol strains, and some have been applied in commercial products (Joseph W. Kloepper, et al. 2004, Phytopathology Vol. 94, No. 11, 1259-1266). For example, strains currently being used in commercial biocontrol products include: Bacillus pumilus strain Q5T2808, used as active ingredient in SONATA® and BALLAD®-PLUS, produced by BAYER CROP SCIENCE; Bacillus pumilus strain GB34, used as active ingredient in YIELDSHIELD, produced by BAYER CROP SCIENCE; Bacillus subtilis strain QO1713, used as the active ingredient of SERENADE™, produced by BAYER CROP SCIENCE; Bacillus subtilis strain GBO3, used as the active ingredient in KODIAK® and SYSTEM3®, produced by HELENA CHEMICAL COMPANY. Various strains of Bacillus thuringiensis and Bacillus firmus have been applied as biocontrol agents against nematodes and vector insects and these strains serve as the basis of numerous commercially available biocontrol products, including NORTICA and PONCHO™-VOTIVO™, produced by BAYER CROP SCIENCE. In addition, Bacillus strains currently being used in commercial biostimulant products include: Bacillus amyloliquefaciens strain FZB42 used as the active ingredient in RHIZOVITAL® 42, produced by ABiTEP GmbH, as well as various other Bacillus subtilus species that are included as whole cells including their fermentation extract in biostimulant products, such as FULZYME produced by JHBiotech Inc.

The presently disclosed subject matter provides microbial compositions and methods for their use in benefiting plant growth as antifungal agents.

### SUMMARY

In one embodiment a plant seed is provided, wherein the plant seed is coated with a composition for controlling plant fungal pathogens, the composition comprising: spores of a biologically pure culture of Bacillus licheniformis CH200 deposited as DSM 17236, or a mutant thereof having all the identifying characteristics thereof; and spores of a biologically pure culture of Bacillus subtilis CH201 deposited as DSM 17231, or a mutant thereof having all the identifying characteristics thereof.

In one embodiment a method is provided for controlling plant fungal pathogens, the method comprising: planting a seed of a plant in a suitable growth medium, wherein the seed has been coated with a composition comprising: spores of a biologically pure culture of Bacillus licheniformis CH200 deposited as DSM 17236, or a mutant thereof having all the identifying characteristics thereof; and spores of a biologically pure culture of Bacillus subtilis CH201 deposited as DSM 17231, or a mutant thereof having all the identifying characteristics thereof, wherein the Bacillus licheniformis CH200 and the Bacillus subtilis CH201 are present in an amount suitable to improve plant yield in the presence of a plant fungal pathogen, improve plant resistance to a plant fungal pathogen, or reduce plant infection by a fungal pathogen, and combinations thereof.

In one embodiment a method is provided for controlling plant fungal pathogens, the method comprising: delivering to seed of a plant, roots of a plant, or soil surrounding a plant a composition comprising: a biologically pure culture of a Bacillus licheniformis CH200 deposited as DSM 17236, or a mutant thereof having all the identifying characteristics thereof; and a biologically pure culture of a Bacillus subtilis CH201 deposited as DSM 17231, or a mutant thereof having all the identifying characteristics thereof, wherein delivery of the composition improves plant yield in the presence of a plant fungal pathogen, improves plant resistance to a plant fungal pathogen, or reduces plant infection by a fungal pathogen, and combinations thereof.

In one embodiment a method is provided for controlling plant fungal pathogens, the method comprising: delivering to seed of a plant, roots of a plant, or soil surrounding a plant a combination of: a first composition comprising a biologically pure culture of a Bacillus licheniformis CH200 deposited as DSM 17236, or a mutant thereof having all the identifying characteristics thereof; and a second composition comprising a biologically pure culture of a Bacillus subtilis CH201 deposited as DSM 17231, or a mutant thereof having all the identifying characteristics thereof, wherein delivery of the combination improves plant yield in the presence of a plant fungal pathogen, improves plant resistance to a plant fungal pathogen, or reduces plant infection by a fungal pathogen, and combinations thereof.

In an alternative embodiment, a method is provided for controlling plant fungal pathogens, the method comprising: delivering to the foliage of a plant a plant a composition comprising: a biologically pure culture of a Bacillus licheniformis CH200 deposited as DSM 17236, or a mutant thereof having all the identifying characteristics thereof; and a biologically pure culture of a Bacillus subtilis CH201 deposited as DSM 17231, or a mutant thereof having all the identifying characteristics thereof, wherein delivery of the composition improves plant yield in the presence of a plant fungal pathogen, improves plant resistance to a plant fungal pathogen, or reduces plant infection by a fungal pathogen, and combinations thereof.

In another alternative embodiment, a method is provided for controlling plant fungal pathogens, the method comprising: delivering to the foliage of a plant a plant a combination of: a first composition comprising a biologically pure culture of a Bacillus licheniformis CH200 deposited as DSM 17236, or a mutant thereof having all the identifying characteristics thereof; and a second composition comprising a biologically pure culture of a Bacillus subtilis CH201 deposited as DSM 17231, or a mutant thereof having all the identifying characteristics thereof, wherein delivery of the combination improves plant yield in the presence of a plant fungal pathogen, improves plant resistance to a plant fungal pathogen, or reduces plant infection by a fungal pathogen, and combinations thereof.

In embodiments, the compositions may contain: (i) a biologically pure culture of a Bacillus licheniformis CH200 deposited as DSM 17236, or a mutant thereof having all the identifying characteristics thereof; (ii) a biologically pure culture of a Bacillus subtilis CH201 deposited as DSM 17231, or a mutant thereof having all the identifying characteristics thereof; and (iii) at least one inactive component selected from the group consisting of carriers, solvents, and surface active agents.

### BRIEF DESCRIPTION OF THE DRAWINGS

FIG. 1 is a schematic diagram showing Fengycin- and Dehydroxyfengycin-type cyclic lipopeptides produced by the Bacillus licheniformis CH200 isolate according to one or more embodiments of the present invention.

### DETAILED DESCRIPTION

The presently disclosed subject matter now will be described more fully hereinafter with reference to the accompanying FIGURES, in which some, but not all embodiments of the presently disclosed subject matter are shown. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims.

Following long-standing patent law convention, the terms “a,” “an,” and “the” refer to “one or more” when used in this application, including the claims. Thus, for example, reference to “a plant” includes a plurality of plants, unless the context dearly is to the contrary, and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 Includes 1, 2, 3, 4, and 5, as well as fractions thereof, e.g., 1.5, 2.25, 3.75, 4.1, and the like) and any range within that range.

In one or more embodiments of the present invention, compositions and methods are provided for benefiting plant growth and conferring protection against or controlling plant fungal pathogenic infection. In one embodiment, a method is provided that includes delivering to seed of a plant, roots of a plant, or soil surrounding a plant a composition comprising: a biologically pure culture of a Bacillus licheniformis CH200 deposited as DSM 17236, or a mutant thereof having all the identifying characteristics thereof; and a biologically pure culture of a Bacillus subtilis CH201 deposited as DSM 17231, or a mutant thereof having all the identifying characteristics thereof, wherein delivery of the composition improves plant yield in the presence of a plant fungal pathogen, improves plant resistance to a plant fungal pathogen, or reduces plant infection by a fungal pathogen, and combinations thereof.

The compositions described herein comprising the microorganisms may further comprise one or a combination of a carrier, a dispersant or a yeast extract.

The present inventors have identified that delivery of this combination of strains of Bacillus licheniformis CH200 and Bacillus subtilis CH201 to plants provides unexpected benefits for controlling plant fungal pathogens and improving plant yield. In particular, the activity of this combination of strains under high fungal pathogen pressure results in significant yield enhancements such as in corn and soybean and is useful in combating Sudden Death Syndrome in soybean. Both strains demonstrate strong to very strong antagonistic activity against the majority of the 13 different plant fungal pathogens tested. The Bacillus licheniformis CH200 strain produces a wide range of fengycin and dehydroxyfengycin molecules, which correlates well with the very strong antifungal activity observed for this strain.

In one embodiment, the phrases “a biologically pure culture of a Bacillus licheniformis CH200” and “a biologically pure culture of a Bacillus subtilis CH201” each individually refer to one or a combination of: spores of a biologically pure fermentation culture of the bacterial strain, vegetative cells of a biologically pure fermentation culture of the bacterial strain, one or more products of a biologically pure fermentation culture of the bacterial strain, a culture solid of a biologically pure fermentation culture of the bacterial strain, a culture supernatant of a biologically pure fermentation culture of the bacterial strain, and an extract of a biologically pure fermentation culture of the bacterial strain.

In another embodiment, the phrases “a biologically pure culture of a Bacillus licheniformis CH200” and “a biologically pure culture of a Bacillus subtilis CH201” each individually refer to one or a combination of: spores of a biologically pure fermentation culture of the bacterial strain, vegetative cells of a biologically pure fermentation culture of the bacterial strain, one or more products of a biologically pure fermentation culture of the bacterial strain, and a culture solid of a biologically pure fermentation culture of the bacterial strain. In one variant of this embodiment, each phrase may refer to the spores of a biologically pure fermentation culture of the bacterial strain.

In still another embodiment, the phrases “a biologically pure culture of a Bacillus licheniformis CH200” and “a biologically pure culture of a Bacillus subtilis CH201” each individually refer to one or a combination of: a culture supernatant of a biologically pure fermentation culture of the bacterial strain, and an extract of a biologically pure fermentation culture of the bacterial strain.

The beneficial activity of this combination of CH200 and CH201 bacterial strains for controlling plant fungal pathogenic infection and increasing plant yield is exemplified by the in vitro and field trial studies described herein at EXAMPLES 1-6.

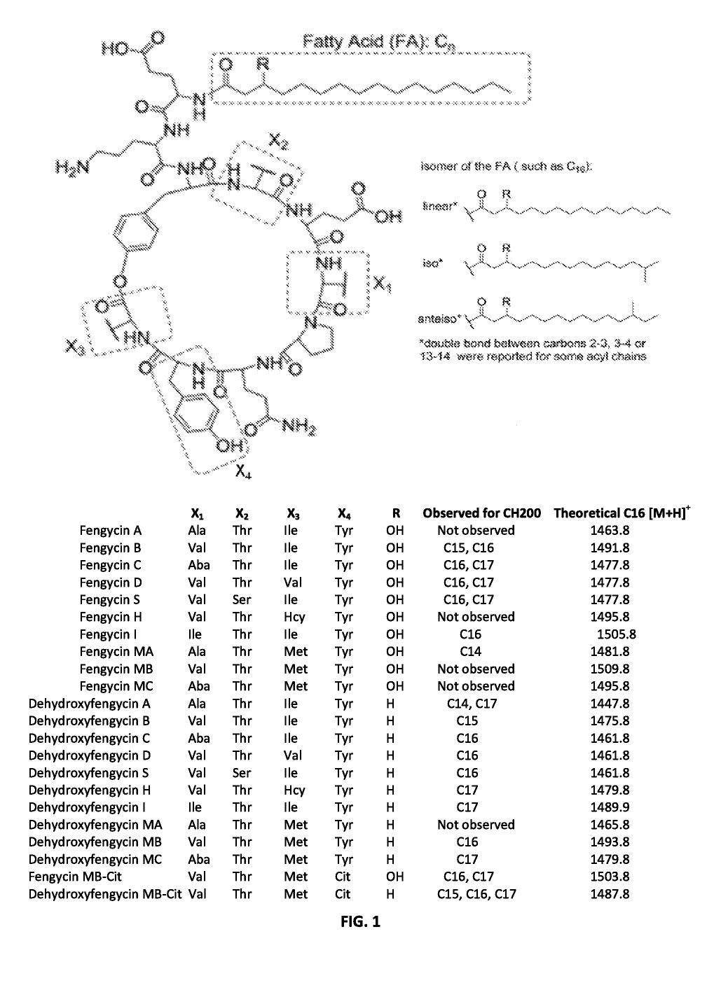

Specifically, EXAMPLE 1 describes the antagonistic activity of each of the Bacillus licheniformis CH200 (Table I) and the Bacillus subtilis CH201 (Table II) isolates against major plant fungal pathogens as measured in plate assays. Both strains demonstrate strong to very strong antagonistic activity against the majority of the 13 different plant fungal pathogens tested, with the CH200 strain being an especially good antifungal agent. In addition, given the very strong anti-fungal activity observed for the CH200 strain, the composition of the antimicrobial Fengycin- and Dehydroxyfengycin-type metabolites produced by this strain was determined using UHPLC-TOF MS and peptide sequencing using LC-MS-MS to determine amino acid composition. FIG. 1 is a schematic diagram showing Fengycin- and Dehydroxyfengycin-type cyclic lipopeptides produced by the Bacillus licheniformis CH200 isolate. As shown in FIG. 1 and Table III, Bacillus licheniformis strain CH200 produces Fengycins B, C, D, and S, but not Fengycin A, and produces all of the Dehydroxyfengycins A, B, C, D, and S. In addition, the CH200 strain produces a class of Dehydroxyfengycin where the L-isoleucine of Dehydroxyfengycin B (position X₃ in FIG. 1) is replaced by L-homo-cysteine (Hcy). This Dehydroxyfengycin metabolite is referred to herein as Dehydroxyfengycin H. The CH200 strain produces an additional class of Fengycin and Dehydroxyfengycin where the amino add at position 4 of the cyclic peptide backbone structure (position X₁ in FIG. 1) is replaced by L-isoleucine. These Fengycin and Dehydroxyfengycin metabolites are referred to herein as Fengycin I and Dehydroxyfengycin I. Further, the CH200 strain produces derivatives where the L-isoleucine at position 8 of the cyclic peptide chain (referred to as X₃ in FIG. 1) is replaced by L-methionine. These classes of Fengycin and Dehydroxyfengycin are referred to as MA, MB and MC. The CH200 strain produces Fengycin MA, but not Fengycin MB or MC and Dehydroxyfengycin MB and MC but not Dehydroxyfengycin MA. Further, the CH200 strain produces derivatives in which the Tyrosine (Tyr) of Fengycin MB and Dehydroxyfengycin MB (position X₄ in FIG. 1) is replaced by the α-amino add, Citruline. This class of Fengycin and Dehydroxyfengycin is referred to herein as Fengycin MB-Cit and Dehydroxyfengycin MB-Cit. In summary, these data demonstrate that the Bacillus licheniformis CH200 strain produces a wide range of fengycin and dehydroxyfengycin molecules, which correlates well with the very strong antifungal activity observed for this strain against the majority of the 13 plant fungal pathogens tested.

EXAMPLE 2 describes field trials in which seed of soybean and corn was treated with the combination of the CH200 and CH201 strains along with one or more commonly used chemical fungicides and/or insecticides. The results are shown in Tables IV-VI. Table IV shows the results of soybeans field trial under Rhizoctonia disease pressure with seed treated with chemical active ingredients AI1 (Thiamethoxam, Mefenoxamat, and Fludioxonil) and a combination of a 1:1 ratio of Bacillus licheniformis CH200 and Bacillus subtilis CH201. The results demonstrate a significant improvement with the application of the combination of the CH200 and CH201 strains as compared to AI1 chemical actives alone for each of percent emergence, disease level, and vigor for all time points tested. In addition, yield is increased by approximately 1.5 bu/acre with application of the combination of the CH200 and CH201 strains as compared to chemical actives alone. Table V shows the results of soybeans field trial under high Rhizoctonia disease pressure with seed treated with chemical active ingredients AI5 (Fludioxonil, Mefenoxam, Thiophanate-methyl, and Thiamethoxam) and varying amounts of a combination of a 1:1 ratio of Bacillus licheniformis CH200 and Bacillus subtilis CH201. The results demonstrate a significant improvement with the application of both rates of the combination of the CH200 and CH201 strains (5×10⁵ CFU/seed and 5×10⁶ CFU/seed) as compared to chemical actives alone for each of percent emergence, disease level, and vigor for all time points tested. In addition, yield is increased by approximately 2.6 and 3.5 bu/acre with application of the combination of the CH200 and CH201 strains (5×10⁵ CFU/seed and 5×10⁶ CFU/seed, respectively) as compared to AI5 chemical actives alone. Table VI shows the results of corn field trial under Rhizoctonia disease pressure with seed treated with chemical active ingredients AI2+AI3+AI4 (Fludioxonil, Metalaxyl, and Clothianidin) and varying amounts of a combination of a 1:1 ratio of Bacillus licheniformis CH200 and Bacillus subtilis CH201. The results demonstrate a significant improvement with the application of all 3 rates of the combination of the CH200 and CH201 strains (5×10⁵ CFU/seed, 5×10⁶ CFU/seed, and 2.5×10⁷ CFU/seed) as compared to AI2+AI3+AI4 chemical actives alone for each of percent emergence, disease level, and vigor for all time points tested. In addition, yield is increased by approximately 10% with application of the combination of the CH200 and CH201 strains as compared to use of the chemical actives alone.

EXAMPLE 3 describes field trials in which seed of soybean was treated with the combination of the CH200 and CH201 strains along with one or more commonly used chemical agents for pathogen control to investigate the effect on emergence, root disease, and yield in soybean in the presence of Rhizoctonia disease pressure. The results in Table VII show that treating with the combination of the Bacillus licheniformis CH200 and Bacillus subtilis CH201 in addition to the chemical active agents results in improvements in percent emergence, level of root disease, and yield over that of the chemical active agents alone. In addition, the combination of CH200 and CH201 outperformed the commercial product CRUISERMAXX®+VIBRANCE® in both controlling root disease and in improved yield. Specifically, the addition of the combination of CH0200 and CH201 resulted in an average increase in yield of 3.1 bushels per acre over that of the chemical active agents alone (from 51.4 to 54.5 bushels per acre). Thus, seed treatment with the combination of CH200 and CH201 provides significant improvement in yields in soybean, even under conditions of severe pathogen pressure.

EXAMPLE 4 describes field trials in which seed of corn was treated with a combination of the CH200 and CH201 strains along with chemical active agents Fludioxonil, Mefenoxam, and Clothianidin (“Chem Control”) for pathogen control to investigate the effect on yield in soil inoculated with Fusarium graminearum, a causal agent of seed rot, seedling bight and root rot of corn. The results in Table VIII show that inoculation of the soybean seed with the combination of the Bacillus licheniformis CH200 and Bacillus subtilis CH201 strains increased emergence and yield and reduced root disease level of corn when compared to seeds that were treated with the Chem Control alone. Specifically, the combination of CH200 and CH0201 in addition to the Chem Control resulted in an average increase in yield of 14.5 bushels per acre in 3 trials in Wisconsin over that of the Chem Control alone.

The strain of Bacillus licheniformis CH200 was deposited on Apr. 7, 2005 at Deutsche Sammlung von Mikroorganismen und Zellkulturen GmbH, Mascheroder Weg 1 b, D-38124 Braunschweig (DSMZ) and given the accession No. DSM 17236. The strain of Bacillus subtilis CH201 was deposited on Apr. 7, 2005 at Deutsche Sammlung von Mikroorganismen und Zellkulturen GmbH, Mascheroder Weg 1 b, D-38124 10 Braunschweig (DSMZ) and given the accession No. DSM 17231.

In one embodiment, a method is provided for controlling plant fungal pathogens, the method comprising: delivering to seed of a plant, roots of a plant, or soil surrounding a plant a composition comprising: a biologically pure culture of a Bacillus licheniformis CH200 deposited as DSM 17236, or a mutant thereof having all the identifying characteristics thereof; and a biologically pure culture of a Bacillus subtilis CH201 deposited as DSM 17231, or a mutant thereof having all the identifying characteristics thereof. In embodiments, delivery of the composition improves plant yield in the presence of a plant fungal pathogen, improves plant resistance to a plant fungal pathogen, or reduces plant infection by a fungal pathogen, and combinations thereof.

In one embodiment, a method is provided for controlling plant fungal pathogens, the method comprising: delivering to seed of a plant, roots of a plant, or soil surrounding a plant a combination of: a first composition comprising a biologically pure culture of a Bacillus licheniformis CH200 deposited as DSM 17236, or a mutant thereof having all the identifying characteristics thereof; and a second composition comprising a biologically pure culture of a Bacillus subtilis CH201 deposited as DSM 17231, or a mutant thereof having all the identifying characteristics thereof. In embodiments, delivery of the combination of the first and second compositions improves plant yield in the presence of a plant fungal pathogen, improves plant resistance to a plant fungal pathogen, or reduces plant infection by a fungal pathogen, and combinations thereof.

In an embodiment, the first and second compositions are in the form of a liquid and each of the Bacillus licheniformis CH200 and the Bacillus subtilis CH201 are present at a concentration of from about 1.0×10⁸ CFU/ml to about 5.0×10¹³ CFU/ml.

In an embodiment, the first and second compositions are in the form of a dust, a dry wettable powder, a spreadable granule, or a dry wettable granule and each of the Bacillus licheniformis CH200 and the Bacillus subtilis CH201 are present in an amount of from about 1.0×10⁸ CFU/g to about 5.0×10¹³ CFU/g.

In an embodiment, the first and second compositions are in the form of an oil dispersion and each of the Bacillus licheniformis CH200 and the Bacillus subtilis CH201 are present at a concentration of from about 1.0×10⁸ CFU/ml to about 5.0×10¹³ CFU/ml.

In embodiments, one or both of the first and second compositions further comprises one or a combination of a microbial, a biological, or a chemical insecticide, fungicide, nematicde, bacteriocide, herbicide, plant extract, plant growth regulator, or fertilizer present in an amount suitable to benefit plant growth and/or to confer protection against a pathogenic infection in the plant.

In the methods for controlling plant fungal pathogens, the fungal pathogen may include, for example, one or more of a rust fungus, a Botrytis spp., a Botrytis cinerea, a Fusarium spp., a Fusarium colmorum, a Fusarium oxysporum, a Phytophthora spp., a Phytophthora capsici, a Rhizoctonia spp., a Rhizoctonia solani, a Magnaporthe oryzae, a Pythium spp., a Pythium aphanidermatum, a Monilinia fructicola, a Glomerella cingulata, a Sclerotinia spp., a Sclerotinia sclerotiorum, a Scerotinia homeocarpa, a Septoria tritici, a Stagonospora nodorum, or a Cercaspora sojina.

In embodiments, the fungal pathogen may include one or more of: a Botrytis spp., a Botrytis cinerea, a Fusarium spp., a Fusarium colmorum, a Fusarium oxysporum, a Phytophthora spp., a Phytophthora capsici, a Rhizoctonia spp., a Rhizoctonia solani, a Magnaporthe oryzae, a Pythium spp., a Pythium aphanidermatum, a Monilinia fructicola, a Glomerella cingulata, a Sclerotinia spp., a Sclerotinia sclerotiorum, or a Sclerotinia homeocarpa.

In the methods for controlling plant fungal pathogens, the plant can be a wide variety of plants including, for example, monocots, dicots, cereals, Corn, Sweet Corn, Popcorn, Seed Corn, Silage Corn, Field Corn, Rice, Wheat, Barley, Sorghum, Asparagus, Berry, Blueberry, Blackberry, Raspberry, Loganberry, Huckleberry, Cranberry, Gooseberry, Elderberry, Currant, Caneberry, Bushberry, Brassica Vegetables, Broccoli, Cabbage, Cauliflower, Brussels Sprouts, Collards, Kale, Mustard Greens, Kohlrabi, Cucurbit Vegetables, Cucumber, Cantaloupe, Melon, Muskmelon, Squash, Watermelon, Pumpkin, Eggplant, Bulb Vegetables, Onion, Garlic, Shallots, Citrus, Orange, Grapefruit, Lemon, Tangerine, Tangelo, Pummelo, Fruiting Vegetables, Pepper, Tomato, Ground Cherry, Tomatillo, Okra, Grape, Herbs/Spices, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Uma beans, Peas, Chick peas, Split peas, Lentils, Oil Seed Crops, Canola, Castor, Coconut, Cotton, Flax, Oil Palm, Olive, Peanut, Rapeseed, Safflower, Sesame, Sunflower, Soybean, Pome Fruit, Apple, Crabapple, Pear, Quince, Mayhaw, Root/Tuber and Corn Vegetables, Carrot, Potato, Sweet Potato, Cassave, Beets, Ginger, Horseradish, Radish, Ginseng Turnip, Stone Fruit, Apricot, Cherry, Nectarine, Peach, Plum, Prune, Strawberry, Tree Nuts, Almond, Pistachio, Pecan, Walnut, Filberts, Chestnut, Cashew, Beechnut, Butternut, Macadamia, Kiwi, Banana, (Blue) Agave, Grass, Turf grass, Ornamental plants, Poinsettia, Hardwood cuttings, Chestnuts, Oak, Maple, Sugarcane, or Sugarbeet.

In certain embodiments, the plant comprises corn, soybean, cotton or peanut.

In some embodiments of the methods and uses for controlling plant fungal pathogens, the plant is soybean or corn and delivery of the composition or the first and second compositions improves plant yield in the presence of a plant fungal pathogen.

An embodiment provides use of a plant seed coated with a composition for controlling plant fungal pathogens, the composition comprising: spores of a biologically pure culture of Bacillus licheniformis CH200 deposited as DSM 17236, or a mutant thereof having all the identifying characteristics thereof; and spores of a biologically pure culture of Bacillus subtilis CH201 deposited as DSM 17231, or a mutant thereof having all the identifying characteristics thereof. In embodiments, the Bacillus licheniformis CH200 and the Bacillus subtilis CH201 are present in the form of spores or vegetative cells.

In embodiments, each of the Bacillus licheniformis CH200 and the Bacillus subtilis CH201 are present in an amount ranging from about 1.0×10² CFU/seed to about 1.0×10⁹ CFU/seed. In embodiments, the plant seed comprises the seed of corn, soybean, cotton or peanut. In embodiments, the plant seed comprises soybean or corn.

In embodiments, the plant fungal pathogen comprises one or more of a rust fungus, a Botrytis spp., a Botrytis cinerea, a Fusarium spp., a Fusarium colmorum, a Fusarium oxysporum, a Phytophthora spp., a Phytophthora capsici, a Rhizoctonia spp., a Rhizoctonia solani, a Magnaporthe oryzae, a Pythium spp., or a Pythium aphanidermatum.

In embodiments, the plant fungal pathogen comprises one or more of a Botrytis spp., a Botrytis cinerea, a Fusarium spp., a Fusarium colmorum, a Fusarium oxysporum, a Phytophthora spp, a Phytophthora capsici, a Rhizoctonia spp., a Rhizoctonia solani, a Magnaporthe oryzae, a Pythium spp., or a Pythium aphanidermatum.

In embodiments, use of a plant seed is provided wherein the seed is further treated with one or a combination of a microbial, a biological, or a chemical insecticide, fungicide, nematicide, bacteriocide, herbicide, plant extract, plant growth regulator, or fertilizer present in an amount suitable to benefit plant growth and/or to confer protection against a pathogenic infection in the plant. In embodiments, the chemical fungicide is one or a combination of mefenoxam, fluopyram, chlorothalonil, thiophanate-methyl, fludioxonil, metalaxyl, or sedaxane. In embodiments, the chemical insecticide is one or a combination of thiamethoxam, pyrethroids, bifenthrin, tefluthrin, zeta-cypermethrin, organophosphates, chlorethoxyphos, chlorpyrifos, tebupirimphos, cyfluthrin, fiproles, fipronil, nicotinoids, or clothianidin. In embodiments, the chemical insecticide comprises chlorantraniliprole, cyantraniliprole or indoxacarb, optionally in combination with imidacloprid, bifenthrin, thiamethoxam, pyrethroids, tefluthrin, zeta-cypermethrin, organophosphates, chlorethoxyphos, chlorpyrifos, tebupirimphos, cyfluthrin, fiproles, fipronil, nicotinods, or clothianidin. In embodiments, the chemical fungicides comprise a combination of fludioxonil, mefenoxam, thiophanate-Methyl, and thiamethoxam.

The compositions comprising the Bacillus licheniformis CH200 and the Bacillus subtilis CH201 microorganisms can be in the form of a liquid, an oil dispersion, a dust, a dry wettable powder, a spreadable granule, or a dry wettable granule. The microorganisms can be present in the form of spores or vegetative cells. The composition can be in the form of a liquid and each of the Bacillus licheniformis CH200 and the Bacillus subtilis CH201 can be present at a concentration of from about 1.0×10⁸ CFU/ml to about 5×10¹³ CFU/ml. The composition can be in the form of a dust, a dry wettable powder, a spreadable granule, or a dry wettable granule and each of the Bacillus licheniformis CH200 and the Bacillus subtilis CH201 can be present in an amount of from about 1.0×10⁸ CFU/g to about 5×10¹³ CFU/g. The composition can be in the form of an oil dispersion and each of the Bacillus licheniformis CH200 and the Bacillus subtilis CH201 can be present at a concentration of from about 1.0×10⁸ CFU/ml to about 5×10¹³ CFU/ml.

In embodiments, the composition comprises by weight percent: 0.5-40% of a biologically pure culture of not less than about 1×10¹¹ CFU/g Bacillus licheniformis CH200 deposited as DSM 17236, or a mutant thereof having all the identifying characteristics thereof; 0.5-40% of a biologically pure culture of not less than about 1×10¹¹ CFU/g Bacillus subtilis CH201 deposited as DSM 17231, or a mutant thereof having all the identifying characteristics thereof; a liquid carrier; a surface active agent; and an adjuvant. In embodiments, the adjuvant may be selected from the group consisting of viscosity modifiers, thickeners, preservatives, biocides or biostatic agents, antifreezes, crystallization inhibitors, suspending agents, dyes, anti-oxidants, foaming agents, light absorbers, mixing auxiliaries, antifoams, complexing agents, neutralizing or pH-modifying substances and buffers, corrosion inhibitors, fragrances, wetting agents, take-up enhancers, micronutrients, plasticizers, glidants, lubricants, and dispersants.

In embodiments, the composition may be a suspension concentrate comprising water and at least one surface active agent, and one or more additional adjuvants. In embodiments, the one or more adjuvants may be selected from thickeners, solvents, preservatives, antifreeze agents, and antifoam agents. Optionally, the suspension concentrate is further diluted with water before delivery of the composition.

In embodiments, the liquid composition is a suspension concentrate comprising from 1 to 10 weight % of the Bacillus licheniformis culture or a mutant thereof having all the identifying characteristics thereof; from 1 to 10 weight % of the Bacillus subtilis culture or a mutant thereof having all the identifying characteristics thereof; 1 to 5 weight % of one or more surface active agent; and at least one thickener, solvent, preservative, antifreeze agent, or antifoam agent each independently comprising up to about 1 weight % of the composition.

In embodiments, the composition can be in the form of a dust, a dry wettable powder, a spreadable granule, or a dry wettable granule and each of the Bacillus licheniformis CH200 and the Bacillus subtilis CH201 can be present in an amount of from about 1.0×10⁸ CFU/g to about 5×10¹³ CFU/g. In embodiments, the composition may comprise a solid carrier selected from the group consisting of mono- or di-saccharides, oligo- or poly-saccharides, talc, titanium dioxide, pyrophyllite clay, attapulgite day, kieselguhr, silica, limestone, bentonite, calcium montmorillonite, sodium, potassium, magnesium, calcium or ammonium salts of acetate, carbonate, chloride, citrate, phosphate, or sulfate, cottonseed husks, wheat flour, soybean flour, pumice, wood flour, ground nut shells, lignin, yeast extracts, fish meal, or mixtures thereof.

In an embodiment, the composition comprises: 5-40% of a biologically pure culture of not less than about 1×10n CFU/g Bacillus licheniformis CH200 deposited as DSM 17236, or a mutant thereof having all the identifying characteristics thereof; 5-40% of a biologically pure culture of not less than about 1×10n CFU/g Bacillus subtilis CH201 deposited as DSM 17231, or a mutant thereof having all the identifying characteristics thereof; and maltodextrin, silica, calcium carbonate, or any mixtures thereof. In embodiments, the composition comprises 5-15% of maltodextrin.

In embodiments, the composition may comprise by weight %: 5-20% of a biologically pure culture of not less than about 1×10¹¹ CFU/g Bacillus licheniformis CH200 deposited as DSM 17236, or a mutant thereof having all the identifying characteristics thereof; 5-20% of a biologically pure culture of not less than about 1×10¹¹ CFU/g Bacillus subtilis CH201 deposited as DSM 17231, or a mutant thereof having all the identifying characteristics thereof; 5-15% maltodextrin; 35-45% calcium carbonate; and 5-15% silica. In embodiments, the composition may be a wettable powder formulation.

In an embodiment, the composition may be a wettable powder formulation comprising by weight %: about 20% of a biologically pure culture of not less than about 1×10¹¹ CFU/g Bacillus licheniformis CH200 deposited as DSM 17236, or a mutant thereof having all the identifying characteristics thereof; 20% of a biologically pure culture of not less than about 1×10¹ CFU/g Bacillus subtilis CH201 deposited as DSM 17231, or a mutant thereof having all the identifying characteristics thereof; 10% maltodextrin; 40% calcium carbonate; and 10% silica.

In embodiments, the composition is useful in either plant seed treatment or in-furrow applications for conferring protection against or controlling plant fungal pathogenic infection. For seed treatment, a solution of the composition can be applied to seed using standard seed treatment procedures. The composition may be applied to untreated seeds or seeds that have been treated with at least one additional crop protection agent as described below. Alternatively, the composition may also be mixed with an additional crop protection agent for seed treatment or in-furrow applications. In some embodiments, the composition may be applied to the foliage of the plant to be protected, optionally mixed with an additional crop protection agent.

In some embodiments of compositions and methods for controlling plant fungal pathogens, the composition or one or both of the first and second compositions further includes one or a combination of a microbial, a biological, or a chemical insecticide, fungicide, nematicde, bacteriocide, herbicide, plant extract, plant growth regulator, or fertilizer present in an amount suitable to benefit plant growth and/or to confer protection against a pathogenic infection in the plant. The chemical fungicide can be one or a combination of mefenoxam, fluopyram, chlorothalonil, thiophanate-methyl, fludioxonil, metalaxyl, or sedaxane. The chemical insecticide can be one or a combination of thiamethoxam, pyrethroids, bifenthrin, tefluthrin, zeta-cypermethrin, organophosphates, chlorethoxyphos, chlorpyrifos, tebupirimphos, cyfluthrin, fiproles, fipronil, nicotinoids, or clothianidin.

In embodiments, the chemical insecticide may comprise chlorantraniliprole, cyantraniliprole, cyclaniliprole or indoxacarb, optionally in combination with imidadoprid, bifenthrin, thiamethoxam, pyrethroids, tefluthrin, zeta-cypermethrin, organophosphates, chlorethoxyphos, chlorpyrifos, tebupirimphos, cyfluthrin, fiproles, fipronil, nicotinoids, or clothianidin.

In one embodiment, the insecticide can include bifenthrin. The nematicide can include cadusafos. The composition can be formulated as a liquid, a powder, a wettable dissolvable granule, or as spreadable granules. The insecticide can include bifenthrin and clothianidin. The insecticide can include bifenthrin and clothianidin and the composition can be formulated for compatibility with a liquid fertilizer. The insecticide can include zeta-cypermethrin.

In some embodiments of the methods for controlling plant fungal pathogens, the composition or one or both of the first and second compositions further includes a bifenthrin insecticide. The composition including the bifenthrin insecticide can be in a formulation compatible with a liquid fertilizer. The formulation compatible with a liquid fertilizer can include a hydrated aluminum-magnesium silicate and at least one dispersant. The bifenthrin insecticide can be present at a concentration ranging from 0.1 g/ml to 0.2 g/ml. The bifenthrin insecticide can be present at a concentration of about 0.1715 g/ml. The term “in a formulation compatible with a liquid fertilizer” as used throughout the specification and claims is intended to mean that the formulation is capable of dissolution or dispersion or emulsion in an aqueous solution to allow for mixing with a fertilizer for delivery to plants in a liquid formulation.

In an embodiment, the bifenthrin composition can include: bifenthrin; a hydrated aluminum-magnesium silicate; and at least one dispersant selected from a sucrose ester, a lignosulfonate, an alkylpolyglycoside, a naphthalenesulfonic acid formaldehyde condensate and a phosphate ester. The bifenthrin can be preferably present in a concentration of from 1.0% by weight to 35% by weight, more particularly, from 15% by weight to 25% by weight based upon the total weight of all components in the composition. The bifenthrin insecticide composition can be present in the liquid formulation at a concentration ranging from 0.1 g/ml to 0.2 g/ml. The bifenthrin insecticide may be present in the liquid formulation at a concentration of about 0.1715 g/ml. The dispersant or dispersants can preferably be present in a total concentration of from about 0.02% by weight to about 20% by weight based upon the total weight of all components in the composition. In some embodiments, the hydrated aluminum-magnesium silicate may be selected from the group consisting of montmorillonite and attapulgite. In some embodiments, the phosphate ester may be selected from a nonyl phenol phosphate ester and a tridecyl alcohol ethoxylated phosphate potassium salt.

Other embodiments may further include at least one of an anti-freeze agent, an anti-foam agent and a biocide.

In some embodiments, the method for controlling plant fungal pathogens can further include applying a liquid fertilizer to: soil or growth medium surrounding the plant or plant seed; or soil or growth medium before sowing the plant in soil or growth medium.

In some embodiments, the method for controlling plant fungal pathogens can further include applying to: the plant or plant seed; soil or growth medium surrounding the plant or plant seed; or soil or growth medium before sowing the plant or plant seed in the soil or growth medium one or more of a microbial, a biological, or a chemical insecticide, fungicide, nematicide, bacteriocide, herbicide, plant extract, or plant growth regulator.

In one embodiment, a plant seed is provided coated with a composition for controlling plant fungal pathogens, the composition comprising: spores of a biologically pure culture of Bacillus licheniformis CH200 deposited as DSM 17236, or a mutant thereof having all the identifying characteristics thereof; and spores of a biologically pure culture of Bacillus subtilis CH201 deposited as DSM 17231, or a mutant thereof having all the identifying characteristics thereof.

In one embodiment, a method is provided for controlling plant fungal pathogens, the method comprising: planting a seed of a plant in a suitable growth medium, wherein the seed has been coated with a composition comprising: spores of a biologically pure culture of Bacillus licheniformis CH200 deposited as DSM 17236, or a mutant thereof having all the identifying characteristics thereof; and spores of a biologically pure culture of Bacillus subtilis CH201 deposited as DSM 17231, or a mutant thereof having all the identifying characteristics thereof, wherein the Bacillus licheniformis CH200 and the Bacillus subtilis CH201 are present in an amount suitable to improve plant yield in the presence of a plant fungal pathogen, improve plant resistance to a plant fungal pathogen, or reduce plant infection by a fungal pathogen, and combinations thereof.

The composition coated onto the plant seed can include an amount of spores of each of the Bacillus licheniformis CH200 and the Bacillus subtilis CH201 from about 1.0×10² CFU/seed to about 1.0×10⁹ CFU/seed.

The fungal pathogens that can be controlled with the composition coated onto the plant seed of the present invention include, for example, a rust fungus, a Botrytis spp., a Botrytis cinerea, a Fusarium spp., a Fusarium colmorum, a Fusarium oxysporum, a Fusarium virguliforme, a Phytophthora spp, a Phytophthora capsici, a Rhizoctonia spp., a Rhizoctonia solani, a Magnaporthe oryzae, a Pythium spp., or a Pythium aphanidermatum.

In embodiments, the plant fungal pathogen comprises one or more of a Botrytis spp., a Botrytis cinerea, a Fusarium spp., a Fusarium colmorum, a Fusarium oxysporum, a Phytophthora spp., a Phytophthora capsici, a Rhizoctonia spp., a Rhizoctonia solani, a Magnaporthe oryzae, a Pythium spp., or a Pythium aphanidermatum.

The coated seed of the present invention can be a seed from a wide variety of plants including for example, the seed of monocots, dicots, cereals, Corn, Sweet Corn, Popcorn, Seed Corn, Silage Corn, Field Corn, Rice, Wheat, Barley, Sorghum, Brassica Vegetables, Broccoli, Cabbage, Cauliflower, Brussels Sprouts, Collards, Kale, Mustard Greens, Kohlrabi, Bulb Vegetables, Onion, Garlic, Shallots, Fruiting Vegetables, Pepper, Tomato, Ground Cherry, Tomatillo, Okra, Grape, Herbs/Spices, Cucurbit Vegetables, Cucumber, Cantaloupe, Melon, Muskmelon, Squash, Watermelon, Pumpkin, Eggplant, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Uma beans, Peas, Chick peas, Split peas, Lentils, Oil Seed Crops, Canola, Castor, Cotton, Flax, Peanut, Rapeseed, Safflower, Sesame, Sunflower, Soybean, Root/Tuber and Corn Vegetables, Carrot, Potato, Sweet Potato, Beets, Ginger, Horseradish, Radish, Ginseng Turnip, Sugarcane, Sugarbeet, Grass, and Turf grass.

In embodiments, the plant seed can be soybean, corn, cotton or peanut; or soybean or corn.

In some embodiments, the method for controlling plant fungal pathogens can further include applying a liquid fertilizer to: soil or growth medium surrounding the plant seed; or soil or growth medium before planting the seed in the soil or growth medium.

In some embodiments, the method for controlling plant fungal pathogens can further include applying to: soil or growth medium surrounding the plant seed; or soil or growth medium before planting the seed in the soil or growth medium one or more of a microbial, a biological, or a chemical insecticide, fungicide, nematicide, bacteriocde, herbicids, plant extract, or plant growth regulator.

In one embodiment, the composition coated onto the plant seed further comprises one or a combination of a microbial, a biological, or a chemical insecticide, fungicide, nematicide, bacteriocide, herbicide, plant extract, plant growth regulator, or fertilizer present in an amount suitable to benefit plant growth and/or to confer protection against a pathogenic infection in the plant.

In one embodiment, the composition coated onto the plant seed includes one or a combination of the chemical fungicides mefenoxam, fluopyram, chlorothalonil, thiophanate-methyl, fludioxonil, metalaxyl, or sedaxane.

In one embodiment, the composition coated onto the plant seed includes one or a combination of the chemical insecticides thiamethoxam, pyrethroids, bifenthrin, tefluthrin, zeta-cypermethrin, organophosphates, chlorethoxyphos, chlorpyrifos, tebupirimphos, cyfluthrin, fiproles, fipronil, nicotinoids, or clothianidin.

In one embodiment, the composition coated onto the plant seed includes a combination of the chemical insecticide and the chemical fungicides fludioxonil, mefenoxam, thiophanate-Methyl, and thiamethoxam.

Suitable insecticides, herbicides, fungicides, and nematicides of the compositions, methods and uses of the present invention can include the following: Insecticides: A0) various insecticides, including agrigata, al-phosphide, amblyselus, aphelinus, aphidius, aphidoletes, artimisinin, autographa californica NPV, azocyclotin, Bacillus subtilis, Bacillus thuringiensis—spp. aizawali, Bacillus thuringiensis spp. kurstaki, Bacillus thuringiensis, Beauveria, Beauvera bossiana, betacyfluthrin, biologicals, bisultap, brofluthrinate, bromophose, bromopropylate, Bt-Corn-GM, Bt-Soya-GM, capsaicin, cartap, celastrus-extract, chlorantraniliprole, chlorbenzuron, chlorethoxyfos, chlorfluazuron, chlorpyrifose, cnidiadin, cryolite, cyanophos, cyantraniliprole, cyclaniliprole, cyhalothrin, cyhexatin, cypermethrin, dacnusa, DCIP, dichloropropene, dicofol, diglyphus, diglyphus+dacnusa, dimethacarb, dithioether, dodecyl-acetate, emamectin, encarsia, EPN, eretmocerus, ethylene-dibromide, eucalyptol, fatty-acids, fatty-acids/salts, fenazaquin, fenobucarb (BPMC), fenpyroximate, flubrocythrinate, flufenzine, formetanate, formothion, furathiocarb, gamma-cyhalothrin, garlic-juice, granulosis-virus, harmonia, heliothis armigera NPV, inactive bacterium, indol-3-ylbutyric add, iodomethane, iron, isocarbofos, isofenphos, isofenphos-m, isoprocarb, isothioate, kaolin, lindane, liuyangmydn, matrine, mephosfolan, metaldehyde, metarhizium-anisopliae, methamidophos, metolcarb (MTMC), mineral-oil, mirex, m-isothiocyanate, monosultap, myrothedum verrucaria, naled, neochrysocharis formosa, nicotine, nicotinolds, oil, oleic acid, omethoate, orius, oxymatrine, paecilomyces, paraffin-oil, parathione, pasteuria, petroleum-oil, pheromones, phosphorus-acid, photorhabdus, phoxim, phytoseiulus, pirimiphose, plant-oil, plutella xylostella GV, polyhedrosis-virus, polyphenol-extracts, potassium-oleate, profenofos, prosuler, prothiofos, pyradofos, pyrethrins, pyridaphenthion, pyrimidifen, pyriproxifen, quillay-extract, quinomethionate, rape-oil, rotenone, saponin, saponozit, sodium-compounds, sodium-fluosilicate, starch, steinemrnema, streptomyces, sulfluramid, sulphur, tebupirimfos, tefluthrin, temephos, tetradifon, thiofanox, thiometon, transgenics (e.g., Cry3Bb1), triazamate, trichoderma, trichogramma, triflumuron, verticillium, vertrine, isomeric insecticides (e.g., kappa-bifenthrin, kappa-tefluthrin), dichloromezotiaz, broflanilide, pyraziflumid; A1) the class of carbamates, including aldicarb, alanycarb, benfuracarb, carbaryl, carbofuran, carbosulfan, methiocarb, methomyl, oxamyl, pirimicarb, propoxur and thiodicarb; A2) the class of organophosphates, including acephate, azinphos-ethyl, azinphos-methyl, chlorfenvinphos, chlorpyrifos, chlorpyrifos-methyl, demeton-S-methyl, diazinon, dichlorvos/DDVP, dicrotophos, dimethoate, disulfoton, ethion, fenitrothion, fenthion, isoxathion, malathion, methamidaphos, methidathion, mevinphos, monocrotophos, oxymethoate, oxydemeton-methyl, parathion, parathion-methyl, phenthoate, phorate, phosalone, phosmet, phosphamidon, pirimiphos-methyl, quinalphos, terbufos, tetrachlorvinphos, triazophos and trichlorfon; A3) the dass of cyclodiene organochlorine compounds such as endosulfan; A4) the class of fiproles, including ethiprole, fipronil, pyrafluprole and pyriprole; A5) the class of neonicotinods, including acetamiprid, dclothianidin, dinotefuran, imidacloprid, nitenpyram, thiacloprid and thiamethoxam; A6) the class of spinosyns such as spinosad and spinetoram; A7) chloride channel activators from the class of mectins, including abamectin, emamectin benzoate, ivermectin, lepimectin and milbemectin; A8) juvenile hormone mimics such as hydroprene, kinoprene, methoprene, fenoxycarb and pyriproxyfen; A9) selective homopteran feeding blockers such as pymetrozine, flonicamid and pyrifluquinazon; A10) mite growth inhibitors such as dofentezine, hexythiazox and etoxazole; A11) inhibitors of mitochondrial ATP synthase such as diafenthiuron, fenbutatin oxide and propargite; uncouplers of oxidative phosphorylation such as chlorfenapyr; A12) nicotinic acetylcholine receptor channel blockers such as bensultap, cartap hydrochloride, thiocyclam and thiosultap sodium; A13) inhibitors of the chitin biosynthesis type 0 from the benzoylurea class, including bistrifluron, diflubenzuron, flufenoxuron, hexaflumuron, lufenuron, novaluron and teflubenzuron; A14) inhibitors of the chitin biosynthesis type 1 such as buprofezin; A15) molting disruptors such as cyromazine; A16) ecdyson receptor agonists such as methoxyfenozide, tebufenozide, halofenozide and chromafenozide; A17) octopamin receptor agonists such as amitraz; A18) mitochondrial complex electron transport inhibitors pyridaben, tebufenpyrad, toifenpyrad, flufenerim, cyenopyrafen, cyflumetofen, hydramethylnon, acequinocyl or fluacrypyrim; A19) voltage-dependent sodium channel blockers such as indoxacarb and metaflumizone; A20) inhibitors of the lipid synthesis such as spirodidofen, spiromesifen and spirotetramat; A21) ryanodine receptor-modulators from the class of diamides, including flubendiamide, the phthalamide compounds (R)-3-Chlor-N1-{2-methyl-4-[1,2,2,2-tetrafluor-1-(trifluormethyl)ethyl]phenyl}-N2-(1-methyl-2-methylsulfonylethyl)phthalamide and (S)-3-Chlor-N1-{2-methyl-4-[1,2,2,2-tetrafluor-1-(trifluormethyl)ethyl]phenyl}-N2-(1-methyl-2-methylsulfonylethyl)phthalamid, chlorantraniliprole, cyclaniliprole and cyantraniliprole; A22) compounds of unknown or uncertain mode of action such as azadirachtin, amidoflumet, bifenazate, fluensulfone, piperonyl butoxide, pyridalyl, sulfoxaflor; or A23) sodium channel modulators from the class of pyrethroids, including acrinathrin, allethrin, bifenthrin, cyfluthrin, lambda-cyhalothrin, cyper-methrin, alpha-cypermethrin, beta-cypermethrin, zeta-cypermethrin, deltamethrin, esfenvalerate, etofenprox, fenpropathrin, fenvalerate, flucythrinate, tau-fluvalinate, permethrin, silafluofen and tralomethrin.

Fungicides: B0) benzovindiflupyr, antiperonosporic agents, ametoctradin, amisulbrom, copper salts (e.g., copper hydroxide, copper oxychloride, copper sulfate, copper persulfate), boscalid, thiflumazide, flutianil, furalaxyl, thiabendazole, benodanil, mepronil, isofetamid, fenfuram, bixafen, fluxapyroxad, penflufen, sedaxane, coumoxystrobin, enoxastrobin, flufenoxystrobin, pyraoxystrobin, pyrametostrobin, triclopyricarb, fenaminstrobin, metominostrobin, pyribencarb, meptyldinocap, fentin acetate, fentin chloride, fentin hydroxide, oxytetracycline, chlozolinate, chloroneb, tecnazene, etridiazole, iodocarb, prothiocarb, Bacillus subtilis syn., Bacillus amyloliquefacens (e.g., strains QST 713, FZB24, MBI600, D747), extract from Melaleuca alternifolia, pyrisoxazole, oxpoconazole, etaconazole, fenpyrazamine, fenpicoxamide, mefentrifluconazole, naftifine, terbinafine, validamycin, pyrimorph, valifenalate, phthalide, probenazole, isotianil, laminarin, estract from Reynoutria sachalinensis, phosphorous add and salts, teclofthalam, triazoxide, pyriofenone, organic oils, potassium bicarbonate, chlorothalonil, fluoroimide; B1) azoles, including bitertanol, bromuconazole, cyproconazole, difenoconazole, diniconazole, eniconazole, epoxiconazole, fluquinconazole, fenbuconazole, flusilazole, flutiafol, hexaconazole, Imibenconazole, Ipconazole, metconazole, myclobutanil, penconazole, propiconazole, prothioconazole, simeconazole, triadimefon, triadimenol, tebuconazole, tetraconazole, triticonazole, prochloraz, pefurazoate, Imazalil, triflumizole, cyazofamid, benomyl, carbendazim, thia-bendazole, fuberidazole, ethaboxam, etridiazole and hymexazole, azaconazole, diniconazole-M, oxpoconazol, padlobutrazol, uniconazol, 1-(4-chloro-phenyl)-2-([1,2,4]triazol-1-yl)-cycloheptanol and imazalilsulfphate; B2) strobilurins, including azoxystrobin, dimoxystrobin, enestroburin, fluoxastrobin, kresoxim-methyl, methominostrobin, orysastrobin, picoxystrobin, pyradostrobin, trifloxystrobin, enestroburin, methyl (2-chloro-5-[1-(3-methenzyloxyimino)ethyl]benzyl)carbamate, methyl (2-chloro-[1-(6-methylpyridin-2-ylmethoxyimino)ethyl]benzyl)carbamate and methyl 2-(ortho-(2,5-dimethylphenyloxymethylene)-phenyl)-3-methoxyacrylate, 2-(2-(6-(3-chloro-2-methyl-phenoxy)-5-fluoro-pyridin-4-yloxy)-phenyl)-2-methoxyimino-N-methyl-acetamide and 3-methoxy-2-(2-(N-(4-methoxy-phenyl)-cyclopropanecarboximidoylsulfanylmethyl)-phenyl)-acrylic acid methyl ester; B3) carboxamides, including carboxin, benalaxyl, benalaxyl-M, fenhexamid, flutolanil, furametpyr, mepronil, metalaxyl, mefenoxam, ofurace, oxadixyl, oxycarboxin, penthiopyrad, isopyrazam, thifluzamide, tiadinil, 3,4-dichloro-N-(2-cyanophenyl)isothiazole-5-carboxamide, dimethomorph, flumorph, flumetover, fluopicolide (picobenzamld), zoxamide, carpropamid, didocymet, mandipropamid, N-(2-(4-[3-(chlorophenyl)prop-2-ynyloxy]-3-methoxyphenyl)ethyl)-2-methanesufonylamino-3-methylbutyramide, N-(2-(4-[3-(chloro-phenyl)prop-2-ynyloxy]-3-methoxyphenyl)ethyl)-2-ethanesulfonylamino-3-methylbutyramide, methyl 3-(chlorophenyl)-3-(2-isopropoxycarbonyl-amino-3-methyl-butyrylamino)propionate, N-(4′-bromobiphenyl-2-yl)-4-difluoromethyl-2-methylthiazole-5-carboxamide, N-(4′-trifluoromethyl-biphenyl-2-yl)-4-difluoromethyl-2-methylthiazole-5-carboxamide, N-(4′-chloro-3′-fluorobiphenyl-2-yl)-4-difluoromethyl-2-methyl-thiazole-5-carboxamide, N-(3′,4′-dichloro-4-fluorobiphenyl-2-yl)-3-difluoro-methyl-1-methyl-pyrazole-4-carboxamide, N-(3′,4′-dichloro-5-fluorobiphenyl-2-yl)-3-difluoromethyl-1-methyl pyrazole-4-carboxamide, N-(2-cyanophenyl)-3,4-dichloroisothiazole-5-carboxamide, 2-amino-4-methyl-thiazole-5-carboxamide, 2-chloro-N-(1,1,3-trimethyl-indan-4-yl)-nicotinamide, N-(2-(1,3-dimethylbutyl)-phenyl)-1,3-dimethyl-5-fluoro-1H-pyrazole-4-carboxamide, N-(4′-chloro-3′,5-difluoro-biphenyl-2-yl)-3-difluoromethyl-1-methyl-1H-pyrazole-4-carboxamide, N-(4′-chloro-3′,5-difluoro-biphenyl-2-yl)-3-trifluoromethyl-1-methyl-1H-pyrazole-4-carboxamide, N-(3,4′-dichloro-5-fluoro-biphenyl-2-yl)-3-trifluoromethyl-1-methyl-1H-pyrazole-4-carboxamide, N-(3′,5-difluoro-4′-methyl-biphenyl-2-yl)-3-difluoromethyl-1-methyl-1H-pyrazole-4-carboxamide, N-(3′,5-difluoro-4′-methyl-biphenyl-2-yl)-3-trifluoromethyl-1-methyl-H-pyrazole-4-carboxamide, N-(cis-2-bicyclopropyl-2-yl-phenyl)-3-difluoromethyl-1-methyl-1H-pyrazole-4-carboxamide, N-(trans-2-bicyclopropyl-2-yl-phenyl)-3-difluoro-methyl-1-methyl-H-pyrazole-4-carboxamide, fluopyram, N-(3-ethyl-3,5-5-trimethyl-cyclohexyl)-3-formylamino-2-hydroxy-benzamide, oxytetracylin, silthiofam, N-(6-methoxy-pyridin-3-yl) cyclopropanecarboxamide, 2-iodo-N-phenyl-benzamide, N-(2-bicyclo-propyl-2-yl-phenyl)-3-difluormethyl-1-methylpyrazol-4-ylcarboxamide, N-(3′,4′,5′-trifluorobiphenyl-2-yl)-1,3-dimethylpyrazol-4-ylcarboxamide, N-(3′,4′,5′-trifluorobiphenyl-2-yl)-1,3-dimethyl-5-fluoropyrazol-4-yl-carboxamide, N-(3′,4′,5′-trifluorobiphenyl-2-yl)-5-chloro-1,3-dimethyl-pyrazol-4-ylcarboxamide, N-(3′,4,5-trifluorobiphenyl-2-yl)-3-fluoromethyl-1-methylpyrazol-4-ylcarboxamide, N-(3′,4′,5′-trifluorobiphenyl-2-yl)-3-(chlorofluoromethyl)-1-methylpyrazol-4-ylcarboxamide, N-(3′,4,5′-trifluorobiphenyl-2-yl)-3-difluoromethyl-1-methylpyrazol-4-ylcarboxamide, N-(3′,4′,5′-trifluorobiphenyl-2-yl)-3-difluoromethyl-5-fluoro-1-methylpyrazol-4-ylcarboxamide, N-(3′,4′,5′-trifluorobiphenyl-2-yl)-5-chloro-3-difluoromethyl-1-methylpyrazol-4-ylcarboxamide, N-(3′,4′,5′-trifluorobiphenyl-2-yl)-3-(chlorodifluoromethyl)-1-methylpyrazol-4-ylcarboxamide, N-(3′,4′,5′-trifluorobiphenyl-2-yl)-1-methyl-3-trifluoromethylpyrazol-4-ylcarboxamide, N-(3′,4,5-trifluorobiphenyl-2-yl)-5-fluoro-1-methyl-3-trifluoromethylpyrazol-4-ylcarboxamide, N-(3′,4,5-trifluorobiphenyl-2-yl)-5-chloro-1-methyl-3-trifluoromethylpyrazol-4-ylcarboxamide, N-(2′,4,5-trifluorobiphenyl-2-yl)-1,3-dimethylpyrazol-4-ylcarboxamide, N-(2′,4,5-trifluorobiphenyl-2-yl)-1,3-dimethyl-5-fluoropyrazol-4-ylcarboxamide, N-(2,4,5′-trifluorobiphenyl-2-yl)-5-chloro-1,3-dimethylpyrazol-4-ylcarboxamide, N-(2,4,5′-trifluorobiphenyl-2-yl)-3-fluoromethyl-1-methylpyrazol-+ylcarboxamide, N-(2,4,5′-trifluorophenyl-2-yl)-3-(chlorofluoromethyl)-1-methylpyrazol-4-ylcarboxamide, N-(2,4,5′-trifluorobiphenyl-2-yl)-3-difluoromethyl-1-methylpyrazol-4-ylcarboxamide, N-(2,4,5′-trifluorobiphenyl-2-yl)-3-difluoromethyl-5-fluoro-1-methylpyrazol-4-ylcarboxamide, N-(2,4,5′-trifluorobiphenyl-2-yl)-5-chloro-3-difluoromethyl-1-methylpyrazol-4-ylcarboxamide, N-(2,4′,5′-trifluorobiphenyl-2-yl)-3-(chlorodifluoromethyl)-1-methylpyrazol-4-ylcarboxamide, N-(2,4,5′-trifluorobiphenyl-2-yl)-1-methyl-3-trifluoromethylpyrazol-4-ylcarboxamide, N-(2,4,5′-trifluorobiphenyl-2-yl)-5-fluoro-1-methyl-3-trifluoromethylpyrazol-4-ylcarboxamide, N-(2,4,5′-trifluorobiphenyl-2-yl)-5-chloro-1-methyl-3-trifluoromethylpyrazol-4-ylcarboxamide, N-(3,4′-dichloro-3-fluorobiphenyl-2-yl)-1-methyl-3-trifluoromethyl-1H-pyrazole-4-carboxamide, N-(3,4′-dichloro-3-fluorobiphenyl-2-yl)-1-methyl-3-difluoromethyl-1H-pyrazole-4-carboxamide, N-(3,4′-difluoro-3-fluorobiphenyl-2-yl)-1-methyl-3-trifluoromethyl-1H-pyrazole-4-carboxamide, N-(3,4′-difluoro-3-fluorobiphenyl-2-yl)-1-methyl-S-difluoromethyl-1H-pyrazole-4-carboxamide, N-(3′-chloro-4′-fluoro-3-fluorobiphenyl-2-yl)-1-methyl-3-difluoromethyl-H-pyrazole-4-carboxamide, N-(3′,4′-dichloro-4-fluorobiphenyl-2-yl)-1-methyl-3-trifluoromethyl-1H-pyrazole-4-carboxamide, N-(3′,4′-difluoro-4-fluorobiphenyl-2-yl)-1-methyl-S-trifluoromethyl-IH-pyrazole-4-carboxamide, N-(3′,4′-dichloro-4-fluorobiphenyl-2-yl)-1-methyl-3-difluoromethyl-1H-pyrazole-4-carboxamide, N-(3′,4′-difluoro-5-fluorobiphenyl-2-yl)-1-methyl-3-difluoromethyl-H-pyrazole-4-carboxamide, N-(3′-chloro-4′-fluoro-4-fluorobiphenyl-2-yl)-1-methyl-S-difluoromethyl-IH-pyrazole-4-carboxamide, N-(3′,4′-dichloro-5-fluorobiphenyl-2-yl)-1-methyl-3-trifluoromethyl-H-pyrazole-4-carboxamide, N-(3′,4′-difluoro-5-fluorobiphenyl-2-yl)-1-methyl-3-trifluoromethyl-H-pyrazole-4-carboxamide, N-(3′,4′-dichloro-5-fluorobiphenyl-2-yl)-1-methyl-S-difluoromethyl-H-pyrazole-carboxamide, N-(3′,4′-difluoro-5-fluorobiphenyl-2-yl)-1-methyl-3-difluoromethyl-1H-pyrazole-4-carboxamide, N-(3′,4′-dichloro-5-fluorobiphenyl-2-yl)-1,3-dimethyl-1H-pyrazole-4-carboxamide, N-(3′-chloro-4′-fluoro-5-fluorobiphenyl-2-yl)-1-methyl-3-difluoromethyl-1H-pyrazole-4-carboxamide, N-(4′-fluoro-4-fluorobiphenyl-2-yl)-1-methyl-3-trifluoromethyl-1H-pyrazole-4-carboxamide, N-(4′-fluoro-5-fluorobiphenyl-2-yl)-1-methyl-3-trifluoromethyl-1H-pyrazole-carboxamide, N-(4′-chloro-5-fluorobiphenyl-2-yl)-1-methyl-3-trifluoromethyl-1H-pyrazole-4-carboxamide, N-(4′-methyl-5-fluorobiphenyl-2-yl)-1-methyl-3-trifluoromethyl-1H-pyrazole-4-carboxamide, N-(4′-fluoro-5-fluorobiphenyl-2-yl)-1,3-dimethyl-1H-pyrazole-4-carboxamide, N-(4′-chloro-5-fluorobiphenyl-2-yl)-1,3-dimethyl-1H-pyrazole-4-carboxamide, N-(4′-methyl-5-fluorobiphenyl-2-yl)-1,3-dimethyl-1H-pyrazole-4-carboxamide, N-(4′-fluoro-6-fluorobiphenyl-2-yl)-1-methyl-3-trifluoromethyl-1H-pyrazole-4-carboxamide, N-(4′-chloro-fluorobiphenyl-2-yl)-1-methyl-3-trifluoromethyl-1H-pyrazole-4-carboxamide, N-[2-(1,1,2,3,3,3-hexafluoropropoxy)-phenyl]-3-difluoromethyl-1-methyl-1H-pyrazole-4-carboxamide, N-[4′-(trifluoromethylthio)-biphenyl-2-yl]-3-difluoromethyl-1-methyl-1H-pyrazole-4-carboxamide and N-[4′-(trifluoromethylthio)-biphenyl-2-yl]-1-methyl-3-trifluoromethyl-1-methyl-1H-pyrazole-4-carboxamide; B4) heterocyclic compounds, including fluazinam, pyrifenox, bupirimate, cyprodinil, fenarimol, ferimzone, mepanipyrim, nuarimol, pyrimethanil, triforine, fenpicdonil, fludioxonil, aldimorph, dodemorph, fenpropimorph, tridemorph, fenpropidin, iprodione, procymidone, vindozolin, famoxadone, fenamidone, octhilinone, probenazole, 5-chloro-7-(4-methyl-piperidin-1-yl)-6-(2,4,6-trifluorophenyl)-[1,2,4]triazolo[1,5-a]pyrimidine, anilazine, diclomezine, pyroquilon, proquinazid, tricylazole, 2-butoxyl-6-iodo-3-propylchromen-4-one, acibenzolar-S-methyl, captafol, captan, dazomet, folpet, fenoxanil, quinoxyfen, N,N-dimethyl-3-(3-bromofluoro-2-methylindole-1-sulfonyl)-[1,2,4]triazole-1-sulfonamide, 5-ethyl-6-octyl-[1,2,4]triazolo[1,5-a]pyrimidin-2,7-diamine, 2,3,5,6-tetrachloro-4-methanesulfonylpyridine, 3,4,5-trichloro-pyridine-2,6-dicarbonitrile, N-(1-(5-bromo-3-chloro-pyridin-2-yl)-ethyl)-2,4-dichloro-nicotinamide, N-((5-bromo-3-chloropyridin-2-yl)-methyl)-2,4-dichloro-nicotinamide, diflumetorim, nitrapyrin, dodemorphacetate, fluoroimid, blasticidin-S, chinomethionat, debacarb, difenzoquat, difenzoquat-methylsulphat, oxolinic acid and piperalin; 85) carbamates, including mancozeb, maneb, metam, methasulphocarb, metiram, ferbam, propineb, thiram, zineb, ziram, diethofencarb, iprovalicarb, benthiavalicarb, propamocarb, propamocarb hydrochlorid, 4-fluorophenyl N-(1-(1-(4-cyanophenyl)-ethanesulfonyl)but-2-yl)carbamate, methyl 3-(4-chloro-phenyl)-3-(2-isopropoxycarbonylamino-3-methyl-butyrylamino)propanoate; or B6) other fungicides, including guanidine, dodine, dodine free base, iminoctadine, guazatine, antibiotics: kasugamycin, oxytetracyclin and its salts, streptomycin, polyoxin, validamycin A, nitrophenyl derivatives: binapacryl, dinocap, dinobuton, sulfur-containing heterocyclyl compounds: dithianon, isoprothiolane, organometallic compounds: fentin salts, organophosphorus compounds: edifenphos, iprobenfos, fosetyl, fosetyl-aluminum, phosphorous acid and its salts, pyrazophos, toldofos-methyl, organochlorine compounds: dichlofluanid, flusulfamide, hexachloro-benzene, phthalide, pencycuron, quintozene, thiophanate, thiophanate-methyl, tolytfluanid, others: cyflufenamid, cymoxanil, dimethirimol, ethirimol, furalaxyl, metrafenone and spiroxamine, guazatine-acetate, iminoctadine-triacetate, iminoctadine-tris(albesilate), kasugamycin hydrochloride hydrate, dichlorophen, pentachlorophenol and its salts, N-(4-chloro-2-nitro-phenyl)-N-ethyl-methyl-benzenesulfonamide, didoran, nitrothal-isopropyl, tecnazen, biphenyl, bronopol, diphenylamine, mildiomycin, oxincopper, prohexadione calcium, N-(cyclopropylmethoxyimino-(6-difluoromethoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluormethyl-4-(3-trimethylsilanyl-propoxy)-phenyl)-N-ethyl-N-methylformamidine and N′-(5-difluoromethyl-2-methyl-4-(3-trimethylsilanyl-propoxy)-phenyl)-N-ethyl-N-methyl formamidine.

Herbicides: C1) acetyl-CoA carboxylase inhibitors (ACC), for example cyclohexenone oxime ethers, such as alloxydim, clethodim, doproxydim, cycloxydim, sethoxydim, tralkoxydim, butroxydim, clefoxydim or tepraloxydim; phenoxyphenoxypropionic esters, such as dodinafop-propargyl, cyhalofop-butyl, didofop-methyl, fenoxaprop-ethyl, fenoxaprop-P-ethyl, fenthiapropethyl, fluazifop-butyl, fluazifop-P-butyl, haloxyfop-ethoxyethyl, haloxyfop-methyl, haloxyfop-P-methyl, isoxapyrifop, propaquizafop, quizalofop-ethyl, quizalofop-P-ethyl or quizalofop-tefuryl; or arylaminopropionic acids, such as flamprop-methyl or flamprop-isopropyl; C2 acetolactate synthase inhibitors (ALS), for example imidazolinones, such as imazapyr, imazaquin, imazamethabenz-methyl (imazame), imazamox, imazapic or imazethapyr; pyrimidyl ethers, such as pyrithiobac-acid, pyrithiobac-sodium, bispyribac-sodium, KIH-6127 or pyribenzoxym; sulfonamides, such as florasulam, flumetsulam or metosulam; or sulfonylureas, such as amidosulfuron, azimsulfuron, bensulfuron-methyl, chlorimuron-ethyl, chlorsulfuron, cinosulfuron, cyclosulfamuron, ethametsulfuron-methyl, ethoxysulfuron, flazasulfuron, halosulfuron-methyl, imazosulfuron, metsulfuron-methyl, nicosulfuron, primisulfuron-methyl, prosulfuron, pyrazosulfuron-ethyl, rimsulfuron, sulfometuron-methyl, thifensulfuron-methyl, triasulfuron, tribenuron-methyl, triflusulfuron-methyl, tritosulfuron, sulfosulfuron, foramsulfuron or iodosulfuron; C3) amides, for example allidochlor (CDAA), benzoylprop-ethyl, bromobutide, chiorthiamid, diphenamid, etobenzanidibenzchlomet), fluthiamide, fosamin or monalide; C4) auxin herbicides, for example pyridinecarboxylic acids, such as dopyralid or pidoram; or 2,4-D or benazolin; C5) auxin transport inhibitors, for example naptalame or diflufenzopyr; C6) carotenoid biosynthesis inhibitors, for example benzofenap, domazone (dimethazone), diflufenican, fluorochloridone, fluridone, pyrazolynate, pyrazoxyfen, isoxaflutole, isoxachlortole, mesotrione, sulcotrione (chlormesulone), ketospiradox, flurtamone, norflurazon or amitrol; C7) enolpyruvylshikimate-3-phosphate synthase inhibitors (EPSPS), for example glyphosate or sulfosate; C8) glutamine synthetase inhibitors, for example bilanafos (blalaphos) or glufosinate-ammonium; C9) lipid biosynthesis inhibitors, for example anilides, such as anilofos or mefenacet; chloroacetanilides, such as dimethenamid, 5-dimethenamid, acetochlor, alachlor, butachlor, butenachlor, diethatyl-ethyl, dimethachlor, metazachlor, metolachlor, S-metolachlor, pretilachlor, propachlor, prynachlor, terbuchlor, thenylchlor or xylachlor; thioureas, such as butylate, cycloate, di-allate, dimepiperate, EPTC, esprocarb, molinate, pebulate, prosulfocarb, thiobencarb (benthiocarb), tri-allate or vemolate; or benfuresate or perfluidone; C10) mitosis inhibitors, for example carbamates, such as asulam, carbetamid, chlorpropham, orbencarb, pronamid (propyzamid), propham or tiocarbazil; dinitroanilines, such as benefin, butralin, dinitramin, ethalfluralin, fluchloralin, oryzalin, pendimethalin, prodiamine or trifluralin; pyridines, such as dithiopyr or thiazopyr; or butamifos, chlorthal-dimethyl (DCPA) or maleic hydrazide; C11) protoporphyrinogen IX oxidase inhibitors, for example diphenyl ethers, such as acifluorfen, adfluorfen-sodium, adonifen, bifenox, chlomitrofen (CNP), ethoxyfen, fluorodifen, fluoroglycofen-ethyl, fomesafen, furyloxyfen, lactofen, nitrofen, nitrofluorfen or oxyfluorfen; oxadiazoles, such as oxadiargyl or oxadiazon; cyclic imides, such as azafenidin, butafenacil, carfentrazone-ethyl, cinidon-ethyl, flumidorac-pentyl, flumioxazin, flumipropyn, flupropacil, fluthiacet-methyl, sulfentrazone or thidiazimin; or pyrazoles, such as ET-751.JV 485 or nipyraclofen; C12) photosynthesis inhibitors, for example propanil, pyridate or pyridafol; benzothiadiazinones, such as bentazone; dinitrophenols, for example bromofenoxim, dinoseb, dinoseb-acetate, dinoterb or DNOC; dipyridylenes, such as cyperquat-chloride, difenzoquat-methylsulfate, diquat or paraquat-dichloride; ureas, such as chlorbromuron, chlorotoluron, difenoxuron, dimefuron, diuron, ethidimuron, fenuron, fluometuron, isoproturon, isouron, linuron, methabenzthiazuron, methazole, metobenzuron, metoxuron, monolinuron, neburon, siduron or tebuthiuron; phenols, such as bromoxynil or ioxynil; chloridazon; triazines, such as ametryn, atrazine, cyanazine, desmein, dimethamethryn, hexazinone, prometon, prometryn, propazine, simazine, simetryn, terbumeton, terbutryn, terbutylazine or trietazine; triazinones, such as metamitron or metribuzin; uradls, such as bromacil, lenacil or terbacil; or biscarbamates, such as desmedipham or phenmedipham; C13) synergists, for example oxiranes, such as tridiphane; C14) as cell wall synthesis inhibitors, for example isoxaben or dichlobenil; C15) various other herbicides, for example dichloropropionic acids, such as dalapon; dihydrobenzofurans, such as ethofumesate; phenylacetic acids, such as chlorfenac (fenac); or aziprotryn, barban, bensulide, benzthiazuron, benzofluor, buminafos, buthidazole, buturon, cafenstrole, chlorbufam, chlorfenprop-methyl, chloroxuron, cinmethylin, cumyluron, cycluron, cyprazine, cyprazole, dibenzyluron, dipropetryn, dymron, eglinazin-ethyl, endothall, ethiozin, flucabazone, fluorbentranil, flupoxam, isocarbamid, isopropalin, karbutilate, mefluidide, monuron, napropamide, napropanilide, nitralin, oxacidomefone, phenisopham, piperophos, procyazine, profluralin, pyributicarb, secbumeton, sulfallate (CDEC), terbucarb, triaziflam, triazofenamid or trimeturon; or their environmentally compatible salts.

Nematicides or bionematicides: Benomyl, doethocarb, aldoxycarb, tirpate, diamidafos, fenamiphos, cadusafos, dichlofenthion, ethoprophos, fensulfothion, fosthiazate, heterophos, isamidofof, isazofos, phosphocarb, thionazin, imicyafos, mecarphon, acetoprole, benclothiaz, chloropicrin, dazomet, fluensulfone, 1,3-dichloropropene (telone), dimethyl disulfide, metam sodium, metam potassium, metam salt (all MITC generators), methyl bromide, biological soil amendments (e.g., mustard seeds, mustard seed extracts), steam fumigation of soil, allyl isothiocyanate (AITC), dimethyl sulfate, furfural (aldehyde).

Suitable plant growth regulators of the present invention include the following: Plant Growth Regulators: D1) Antlauxins, such as dofibric acid, 2,3,5-tri-iodobenzoic acid; D2) Auxins such as 4-CPA, 2,4-D, 2,4-DB, 2,4-DEP, dichlorprop, fenoprop, IAA, IBA, naphthaleneacetamide, α-naphthaleneacetic acids, 1-naphthol, naphthoxyacetic acids, potassium naphthenate, sodium naphthenate, 2,4,5-T; D3) cytokinins, such as 2iP, benzyladenine, 4-hydroxyphenethyl alcohol, kinetin, zeatin; D4) defoliants, such as calcium cyanamide, dimethipin, endothal, ethephon, merphos, metoxuron, pentachlorophenol, thidiazuron, tribufos; DS) ethylene inhibitors, such as aviglycine, 1-methylcyclopropene; D6) ethylene releasers, such as ACC, etacelasil, ethephon, glyoxime; D7) gametocides, such as fenridazon, maleic hydrazide; D8) gibberellins, such as gibberellins, gibberellic acid; D9) growth inhibitors, such as abscisic acid, ancymidol, butralin, carbaryl, chlorphonium, chlorpropham, dikegulac, flumetralin, fluoridamid, fosamine, glyphosine, isopyrimol, jasmonic acid, maleic hydrazide, mepiquat, piproctanyl, prohydrojasmon, propham, tiaojiean, 2,3,5-tri-iodobenzoic acid; D10) morphactins, such as chlorfluren, chlorflurenol, dichlorflurenol, flurenol; D11) growth retardants, such as chlormequat, daminozide, flurprimidol, mefluidide, paclobutrazol, tetcyclads, uniconazole; D12) growth stimulators, such as brassinolide, brassinolide-ethyl, DCPTA, forchlorfenuron, hymexazol, prosuler, triacontanol; D13) unclassified plant growth regulators, such as bachmedesh, benzofluor, buminafos, carvone, choline chloride, ciobutide, clofencet, cyanamide, cyclanilide, cycloheximide, cyprosulfamide, epocholeone, ethychlozate, ethylene, fuphenthiourea, furalane, heptopargil, holosulf, inabenfide, karetazan, lead arsenate, methasulfocarb, prohexadione, pydanon, sintofen, triapenthenol, and trinexapac.

In embodiments, suitable insecticides may include ryanodine receptor-modulators, including, but not limited to, chlorantraniliprole, cyclaniliprole or cyantraniliprole, and/or voltage-dependent sodium channel blockers such as indoxacarb, optionally in combination with imidacloprid, bifenthrin, thiamethoxam, pyrethroids, tefluthrin, zeta-cypermethrin, organophosphates, chlorethoxyphos, chlorpyrifos, tebupirimphos, cyfluthrin, fiproles, fipronil, nicotinods, and clothianidin.

In embodiments, suitable insecticides may include Bacillus subtilis, Bacillus thuringiensis—spp. airozawaii, Bacillus thuringiensis spp. kurstaki, Bacillus thuringiensis, chlorantraniliprole, chlorethoxyfos, chlorpyrifose, cyantraniliprole, cyclaniliprole, cypermethrin, dichloropropene, flupyradifurone, gamma-cyhalothrin, profenofos, tebupirimfos, tefluthrin, tetraniliprole, kappa-bifenthrin, kappa-tefluthrin, carbofuran, carbosulfan, oxamyl, thiodicarb; chlorpyrifos, chlorpyrifos-methyl, diazinon, phorate, terbufos, fipronil, acetamiprid, clothianidin, imidacloprid, thiacloprid, thiamethoxam; abamectin, flonicamid, flubendiamide, bifenthrin, lambda-cyhalothrin, cyper-methrin, zeta-cypermethrin, and deltamethrin.

In certain embodiments, suitable insecticides may include clothianidin, thiamethoxam, imidacloprid, tefluthrin, fipronil, chlorpyrifose, bifenthrin, cypermethrin, tebupirimfos, zeta-cypermethrin, gamma-cyhalothrin, oxamyl, cadusafos, chlorantraniliprole, cyantraniliprole, cyclaniliprole, and tetraniliprole.

In embodiments, suitable fungicides may include antiperonosporic compounds, including, but not limited to: ametoctradin, amisulbrom, benthiavalicarb, cyazofamid, cymoxanil, dimethomorph, ethaboxam, famoxadone, fenamidone, flumetover, flumorph, fluopicolide, iprovalicarb, mandipropamid, valifenalate, benalaxyl, benalaxyl-M, furalaxyl, metalaxyl, and metalaxyl-M.

In embodiments, suitable fungicides may include thiabendazole, fluxapyroxad, penflufen, sedaxane, Bacillus subtilis syn., Bacillus amyloliquefacens (e.g., strains QST 713, FZB24, MBI600, D747), bitertanol, cyproconazole, difenoconazole, fluquinconazole, flutriafol, ipconazole, myclobutanil, prothioconazole, triadimefon, triadimenol, tebuconazole, triticonazole, prochloraz, imazalil, benomyl, carbendazim, hymexazole, azoxystrobin, fluoxastrobin, pyradostrobin, trifloxystrobin, carboxin, flutolanil, metalaxyl, mefenoxam, penthiopyrad, fluopyram, silthiofam, fluazinam, pyrimethanil, fludioxonil, iprodione, tricyclazole, captan, dazomet, mancozeb, metam, thiram, guazatine, toldofos-methyl, pencycuron, thiophanate-methyl, fenpicoxamide, and mefentrifluconazole.

In certain embodiments, suitable fungicides may include fludioxonil, prothioconazole, mefenoxam, metalaxyl, tebuconazole, difenoconazole, thiram, carboxin, carbendazim, triticonazole, pencycuron, imazalil, pyradostrobin, sedaxane, trifloxystrobin, fluquinconazole, fluoxastrobin, azoxystrobin, flutriafol, fluxapyroxad, penthiopyrad, fenpicoxamide, and mefentrifluconazole.

In embodiments, the fungicide is one or a combination of fenpicoxamide, mefentrifluconazole, mefenoxam, fluopyram, chlorothalonil, thiophanate-methyl, fludioxonil, metalaxyl, or sedaxane.

In embodiments, suitable nematicides or bionematicides may include: Benomyl, fenamiphos, cadusafos, ethoprophos, fosthiazate, chloropicrin, dazomet, fluensulfone, 1,3-dichloropropene (telone), metam sodium, metam potassium, metam salt (all MITC generators), methyl bromide, allyl isothiocyanate (AITC), fluazaindolizine (DPX-Q8U80), and tioxazafen.

In ceratin embodiments, suitable nematicides may include cadusafos, ethoprophos, fosthiazate, fluensulfone, oxamyl, fluazaindolizine (DPX-Q8U80), and tioxazafen.

The fertilizer can be a liquid fertilizer. The term “liquid fertilizer” refers to a fertilizer in a fluid or liquid form containing various ratios of nitrogen, phosphorous and potassium (for example, but not limited to, 10% nitrogen, 34% phosphorous and 0% potassium) and micronutrients, commonly known as starter fertilizers that are high in phosphorus and promote rapid and vigorous root growth.

Chemical formulations useful for combining with the described biological formulations can be in any appropriate conventional form, for example an emulsion concentrate (EC), a suspension concentrate (SC), a suspo-emulsion (SE), a capsule suspension (CS), a water dispersible granule (WG), an emulsifiable granule (EG), a water in oil emulsion (EO), an oil in water emulsion (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a dispersible concentrate (DC), a wettable powder (WP) or any technically feasible formulation in combination with agriculturally acceptable adjuvants.

In another aspect, the compositions may contain: (i) Bacillus licheniformis as described herein; (ii) Bacillus subtilis as described herein; and (iii) at least one adjuvant inactive component selected from the group consisting of carriers and adjuvants.

Carriers can be liquid or solid. Adjuvants that may be used in such formulations include surface active agents, viscosity modifiers such as thickeners, preservatives, biocides or biostatic agents, antifreezes, crystallization inhibitors, suspending agents, dyes, anti-oxidants, foaming agents, light absorbers, mixing auxiliaries, antifoams, complexing agents, neutralizing or pH-modifying substances and buffers, corrosion inhibitors, fragrances, wetting agents, take-up enhancers, micronutrients, plasticizers, glidants, lubricants, dispersants, and also liquid and solid fertilizers.

In embodiments, the compositions of this invention may be formulated as a suspension concentrate (SC), wettable powder (WP) or wettable granule (WG). Other formulations types include water disperable powders for slurry treatment (WS), oil dispersions (OD), granules for broadcast applications (GR), capsule suspensions (CS), emulsifiable concentrates (EC), emulsions in water (EW), soluble concentrates (SL), mixed formulations of a CS and an SC (ZC), mixed formulations of an SC and an EW as suspo-emulsions (SE), and mixed formulations of a CS and an EW (ZW).

In embodiments, the composition is in the form of an oil dispersion and each of the Bacillus licheniformis CH200 and the Bacillus subtilis CH201 are present at a concentration of from about 1.0×10⁸ CFU/ml to about 5.0×10¹³ CFU/ml.

In embodiments, the compositions can further contain one or more adjuvants and/or carriers. In at least one embodiment, the active ingredients comprising the bacillus species are present in total concentrations ranging between 0.5% to about 95 weight % of the agricultural composition, such as wherein each bacillus species is present in an amount independently selected from a lower limit of 1, 2, 3,4 or 5, 7,8, or 10 weight % to an upper limit of 10, 15, 20, 25, 40, 50, 60, 70, 80 or 90 weight % of the total composition. In another embodiment, agriculturally acceptable carriers constitute about 1% to about 98.5%, such as from a lower limit of 1, 2, 3, 4 or 5 weight % to an upper limit of 10, 15, 20, 25, 40, 50, 60, 70, 80 or 90 weight % of the total composition.

Adjuvants may include preservatives, biocides or biostatic agents, surfactants, thickeners, antifoams, antifreezes.

Surface active agents including surfactants, dispersants and emulsifiers, viscosity enhancing agents, solvents and other adjuvants independently may constitute between about 0.1% to about 25% of the final formulation by weight.

Liquid carriers include solvents and co-solvents including water, petroleum ether, vegetable oils, add anhydrides, amyl acetate, butylene carbonate, cyclohexane, cyclohexanol, diacetone alcohol, 1,2-dichloropropane, diethanolamine, diethylene glycol, diethylene glycol abietate, diethylene glycol butyl ether, diethylene glycol ethyl ether, diethylene glycol methyl ether, 1,4-dioxane, dipropylene glycol, dipropylene glycol methyl ether, dipropylene glycol dibenzoate, diproxitol, alkylpyrrolidone, 2-ethylhexanol, ethylene carbonate, 1,1,1-trichloroethane, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isobornyl acetate, isooctane, isophorone, isopropyl myristate, lactic add, laurylamine, mesityl oxide, methoxypropanol, methyl laurate, methyl octanoate, methyl oleate, methylene chloride, n-hexane, n-octylamine, octadecanoic acid, octylamine acetate, oleic add, oleylamine, polyethylene glycol (PEG), propionic add, propyl lactate, propylene carbonate, propylene glycol, propylene glycol methyl ether, triethyl phosphate, triethylene glycol, xylenesulfonic acid, paraffin, mineral oil, trichloroethylene, perchloroethylene, alcohols of higher molecular weight, such as amyl alcohol, tetrahydrofurfuryl alcohol, hexanol, octanol, liquid amides such as N,N-dimethyloctanamide, N,N-dimethyldecanamide, N-methyl-N-(2-propylheptyl)-acetamide, N-methyl-N-(2-propylheptyl)-formamide, N-methyl-2-pyrrolidone and the like. Preferably, liquid carriers are such that the biological active agents remain essentially unchanged in the composition until after it is applied to the locus of control. Water is generally the carrier of choice for diluting the concentrated formulations.

Suitable solid carriers include, for example, carbohydrates including mono or di carbohydrates such as sucrose, oligo or poly-saccharides such as maltodextrin or pectin, talc, titanium dioxide, pyrophyllite day, attapulgite clay, kleselguhr, silica (silicon dioxide), limestone, bentonite, calcium montmorillonite water soluble salts such as sodium, potassium, magnesium, calcium or ammonium salts of acetate, carbonate, chloride, citrate, phosphate, or sulfate such as calcium carbonate, cottonseed husks, wheat flour, soybean flour, pumice, wood flour, ground walnut shells, lignin and similar substances, yeast extracts, fish meal, or mixtures thereof. Notable solid carriers include maltodextrin, silica, calcium carbonate, or any mixtures thereof.

The compositions may contain a surface-active substance (surfactants, dispersants and emulsifiers) from a very large variety of substances known in the art that are also commercially available. Surface-active substances (described herein generally as surfactants) may be anionic, cationic, non-Ionic or polymeric and they can be used as surfactants, dispersants, emulsifiers, wetting agents or suspending agents or for other purposes.

Surfactants belong to different classes such as cationic surfactants, anionic surfactants, non-ionic surfactants, ionic surfactants, and amphoteric surfactants. According to the invention, the surfactant can be any surfactant or combination of two or more surfactants useful to disperse the biological active ingredients in the formulation or tank mix for application. The amounts of the surfactant in the compositions of this invention may range from about 1 to about 15%, or about 1 to about 10%, preferably about 3 to about 8%, and more preferably about 5 to about 7% w/w.

Examples of some preferred surfactants include cationic, non-Ionic, anionic and/or amphoteric surfactants.

Non-ionic surfactants suitable for this invention include ethoxylated linear alcohols, ethoxylated alkyl phenol, alkyl EO/PO copolymer, polyalkylene glycol monobutyl ether ethoxylated fatty adds/oils, sorbitan laurate, polysorbate, sorbitan oleate, ethoxylated fatty acid alcohols, or alkyl phenols, alkanolamides or alkyloamides (such as diethanolamide, lauric add monoisopropanolamide, and ethoxylated myristamide), xyethylene fatty add esters, polyoxyethylene fatty alcohol ethers (such as alkylaryl polyglycol ethers), alkylphenol/alkylene oxide addition products, such as nonylphenol ethoxylate; alcohol/alkylene oxide addition products, such as tridecylalcohol ethoxylate.

Anionic surfactants include alkyl-, alkylaryl- and arylsulfonates or salts thereof (such as sodium, potassium or calcium salts of lauryl sarcosinate, alkylbenzenesulfonate, dodecylbenzenesulfonate, alkylnaphthalenesulfonates such as dibutylnaphthalenesulfonate, or C₁₄₋₁₆olefin sulfonates), alkyl-, alkylaryl- and arylsulfates or salts thereof (such as sodium, potassium or calcium salts of tridedeth sulfate, lauryl sulfate, decyl sulfate, and diethanolammonium lauryl sulfate) protein hydrolysates, derivatives of polycarboxylic add (such as ammonium lauryl ether carboxylate), olefin sulfonates (such as sodium alpha olefin sulfonate), sarcosinates (such as ammonium cyclohexyl palmitoyl taurinate), succinates (such as disodium N-octadecyl sulfosuccinamate), phosphorus derivatives (such as phosphoric add esters and their equivalent salts).

Cationic surfactants include alkylbenzyltrimethylammonium chloride, ammonium lauryl sulfate and lauramine oxide.

Other surface active substances include soaps, such as sodium stearate; dialkyl esters of sulfosuccinate salts, such as sodium di(2-ethylhexyl)sulfosuccinate; sorbitol esters, such as sorbitol oleate; quaternary amines, such as laurytrimethylammonium chloride, polyethylene glycol esters of fatty acids, such as polyethylene glycol stearate; block copolymers of ethylene oxide and propylene oxide; and salts of mono- and di-alkylphosphate esters.

Also suitable are silicone surfactants, especially polyalkyl-oxide-modified heptamethyltriloxanes which are commercially available e.g. as Silwet L-77®, and also perfluorinated surfactants. Of these, some even more specific types of preferred surfactants include non-ionic linear or branched alcohol ethoxylate surfactants, anionic phosphoric acid ester surfactants (sometimes referred to as “phosphate ester” surfactants), and cationic ethoxylated tallow amine surfactants.

In another aspect, the composition may contain a thickener. Suitable thickeners are rice, starch, gum arabic, gum tragacanth, guar flour, British gum, starch ethers and starch esters, gum resins, galactomannans, magnesium aluminum silicate, xanthan gum, carrageenan, cellulose derivatives, methyl cellulose, carboxymethylcellulose, alginates and combinations thereof. Other known commercial products may include Lattice NTC 50, Lattice NTC 60, methocel, clay, and veegum silica.

In another embodiment, the compositions of this invention may contain an antifreeze agent such as glycerine, ethylene glycol, propylene glycol, urea, calcium chloride, sodium nitrate, magnesium chloride and ammonium sulfate.

Suitable preservatives include but are not limited to C₁₂ to C₁₅ alkyl benzoates, alkyl p-hydroxybenzoates, aloe vera extract, ascorbic acid, benzalkonium chloride, benzoic acid, benzoic add esters of C₉ to C₁₅ alcohols, butylated hydroxytoluene, butylated hydroxyanisole, tert-butylhydroquinone, castor oil, cetyl alcohols, chlorocresol, citric acid, cocoa butter, coconut oil, diazolidinyl urea, diisopropyl adipate, dimethyl polysiloxane, DMDM hydantoin, ethanol, ethylenediaminetetraacetic acid, fatty acids, fatty alcohols, hexadecyl alcohol, hydroxybenzoate esters, iodopropynyl butylcarbamate, isononyl iso-nonanoate, jojoba oil, lanolin oil, mineral oil, oleic add, olive oil, parabens, polyethers, polyoxypropylene butyl ether, polyoxypropylene cetyl ether, potassium sorbate, propyl gallate, silicone oils, sodium propionate, sodium benzoate, sodium bisulfite, sorbic acid, stearic fatty add, sulfur dioxide, vitamin E, vitamin E acetate and derivatives, esters, salts and mixtures thereof. Preferred preservatives include sodium o-phenylphenate, 5-chloro-2-methyl-4-isothiazolin-3-one, 2-methyl-4-isothiazolin-3-one, and 1,2-benisothiazolin-3-one.

Antifoam agents such as XIameter AFE-100, Dow Corning AFs, Dow Corning 1520, 1530, or 1540 may also be used in the presently claimed formulations.

In another aspect, the compositions may be prepared by a process following the steps of combining the biological active ingredients in effective amounts with carriers and adjuvants as described herein. The formulated compositions can be prepared e.g. by mixing the biological active agents with the formulation components in order to obtain compositions in the form of finely divided solids, granules or dispersions. The active ingredients can also be formulated with other components, such as finely divided solids, mineral oils, oils of vegetable or animal origin, modified oils of vegetable or animal origin, organic solvents, water, surface-active substances or combinations thereof.

In some embodiments, the components of the formulation can be dry mixed, or solid and liquid components may be blended together in a homogenizer or other suitable mixing vessel. Simple mixing of the Ingredients by homogenization may be preferable to any form of grinding. In other embodiments, the mixture may further undergo a milling process, such as dry milling or wet milling, until suitable particle sizes ranging from about 1 to about 250 microns are obtained. The composition may have particle sizes of less than 250, less than 100 or preferably less than 50 microns. In a preferred embodiment, the mixture is milled until 90% of the particle size (D90) is less than about 50 microns.

One embodiment is directed to a composition comprising: the biological active ingredients; and iii) at least one formulation component selected from the group consisting of adjuvants for an SC formulation; adjuvants for a WP formulation; and adjuvants for a WG formulation.

In another embodiment, the composition is in the form of an SC, such as one comprising water and at least one surfactant, and one or more additional adjuvants selected from thickeners, solvents, preservatives, antifreeze agents, pH-modifiers, and antifoam agents.

In an embodiment, the SC comprises from 1 to 10 weight % of Bacillus licheniformis as described herein; from 1 to 10 weight % of Bacillus subtilis as described herein; i to 5 weight % of one or more surfactants; and optionally at least one thickener, solvent, preservative, antifreeze agent, or antifoam agent; and water. The optional thickener, solvent, preservative, antifreeze agent, or antifoam agent may each independently comprise up to about 1 weight % of the SC formulation. The SC comprises water in a complementary amount to all the other components to bring the total composition to 100 weight % (qs).

The composition may be in solid form, for example a WP or WG formulation. These formulations comprise at least one solid carrier as described above. In embodiments, WP or WG formulations may comprise from about 1 to about 30 weight %, such as from 1 to 10, 5 to 10, or 5 to 30, or 7 to 30, or 10 to 30 weight %, of Bacillus licheniformis as described herein; from about 1 to about 30 weight %, such as from 1 to 10, 5 to 10, or 5 to 30 or 7 to 30, or 10 to 30 weight %, of Bacillus subtilis as described herein; and at least one solid carrier selected from the group consisting of maltodextrin, calcium carbonate and silica.

In an embodiment, the composition may comprise from 5 to 10 weight % of Bacillus licheniformis as described herein; from 5 to 10 weight % of Bacillus subtilis as described herein; from about 80 to about 90 weight % of maltodextrin, and about 0.5 to about 2 weight % of silica.

In another embodiment, the composition may comprise from 5 to 30 (such as 20%) weight % of Bacillus licheniformis as described herein; from 5 to 30 (such as 20%) weight % of Bacillus subtilis as described herein; from about 30 to about 50 (such as 40%) weight % of maltodextrin, about 10 to 20 (such as 16%) weight % of calcium carbonate and about 0.5 to about 5 (such as 4%) weight % of silica.

In another embodiment, the composition comprises a wettable powder formulation comprising by weight %:

5-30% (such as 20%) of a biologically pure culture of not less than about 1×10¹⁰ CFU/g Bacillus licheniformis CH200 deposited as DSM 17236, or a mutant thereof having all the identifying characteristics thereof;
5-30% (such as 20%) of a biologically pure culture of not less than about 1×10¹⁰ CFU/g Bacillus subtilis CH201 deposited as DSM 17231, or a mutant thereof having all the identifying characteristics thereof;
5-15% (such as 10%) maltodextrin;
35-45% (such as 40%) calcium carbonate; and
5-15% (such as 10%) silica.

The composition may be useful in either plant seed treatment or in-furrow applications. For seed treatment, a solution, slurry, paste, gel or moistened solid of the composition can be applied to seed using standard seed treatment procedures. The composition may be applied to untreated seeds or seeds that have been treated with at least one additional crop protection agent as described herein. Alternatively, the composition may also be mixed with an additional crop protection agent for seed treatment or in-furrow applications.

In furrow applications can include treating the soil in the furrow, preferentially in proximity to the crops seeds at the time of planting, and incorporating the formulation into the soil. In furrow applications can include liquid or solid formulations.

In embodiments, the formulated compositions can be in the form of concentrates that are diluted prior to use, although ready-to-use formulations can also be made. Whereas commercial products will preferably be formulated as concentrates, the end user will normally employ dilute formulations for application to the soil or plant. The dilutions can be made, for example, with water, liquid fertilizers, micronutrients, biological organisms, oil or solvents.

In embodiments, the formulated compositions may additionally include an additive comprising an oil of vegetable or animal origin, a mineral oil, alkyl esters of such oils or mixtures of such oils and oil derivatives. The amount of oil additive in the composition according to the invention is generally from 0.01 to 10%, based on the spray mixture. For example, the oil additive can be added to the spray tank in the desired concentration after the spray mixture has been prepared.

In embodiments, oil additives may comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow.

Where features or aspects of the invention are described in terms of a Markush group or other grouping of alternatives, those skilled in the art will recognized that the invention is also thereby described in terms of any individual member or subgroup of members of the Markush group or other group.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### Anti-Microbial Properties of Bacillus Licheniformis CH200 and Bacillus Subtilis CH0201 Isolates

The antagonistic ability of the Bacillus licheniformis CH200 and Bacillus Subtilis CH201 isolates against major plant pathogens was measured in plate assays. A plate assay for evaluation of antagonism against plant fungal pathogens was performed by growing the bacterial isolate and pathogenic fungi side by side on 869 agar plates at a distance of 4 cm. Plates were incubated at room temperature and checked regularly for up to two weeks for growth behaviors such as growth inhibition, niche occupation, or no effect. In the case of screening for antagonistic properties against bacterial pathogens, the pathogen was first spread as a lawn separately on both 869 agar and PDA plates. Subsequently, 20 μl aliquots of a culture of each of the isolates were spotted on the plates. Plates were incubated at room temperature and checked regularly for up to two weeks for an inhibition zone in the lawn around the positions where Bacillus Licheniformis CH200 and Bacillus Subtilis CH201 had been applied. A summary of the antagonism activity is shown in Table I below for each of the CH200 and CH201 strains, respectively.

TABLE I
Antagonistic properties of Bacillus Licheniformis CH200 and Bacillus
Subtilis CH201 isolates against major plant pathogens

| Anti-Microbial Assays | CH200* | CH201* |
|---|---|---|
| Alternaria solani | +++/+++ | ++/++ |
| Botrytis cinerea | +++/+++ | +++/+++ |
| Cercospora sojina | +++/+++ | +++/+ |
| Fusarium colmorum | +++/+++ | +++/+ |
| Fusarium oxysporum | +++/+++ | ++/++ |
| Fusarium virguliforme | +/++ | +−/+− |
| Glomerella cingulata | +++/+++ | +++/+++ |
| Magnaporthe oryzae | +++/+++ | +++/+++ |
| Monilina fructicola | +++/+++ | +++/++ |
| Rhizoctonia solani | +++/+ | +++/+++ |
| Sclerotinia homeocarpa | +++/− | +++/+− |
| Sclerotinia sclerotiorum | +++/+++ | +++/+++ |
| Septoria tritici | −/+− | +−/+− |
| Stagonospora nodorum | +++/+++ | +++/++ |
| Phytophthora capsici | +++/+++ | +++/+ |
| Pythium aphanidermatum | +/− | ++/+− |

+++ very strong activity,
++ strong activity,
+ activity,
+− weak activity,
− no activity observed
*The symbols to the left correspond to the 869 agar plates and those on the right to the PDA plates.

Given the strong anti-fungal activity observed for the CH200 strain, the composition of the antimicrobial Fengycin- and Dehydroxyfengycin-type metabolites produced by this strain was determined. It has been previously reported that five classes of Fengycin-type metabolites and Dehydroxyfengycin-type metabolites are produced by microbial species including Bacillus licheniformis (U, Xing-Yu, et al., 2013, J. Microbiol. Biotechnol. 23(3), 313-321; Pecci Y, et al. 2010, Mass Spectrom., 45(7):772-77). These metabolites, belonging to the class of cyclic lipopeptides, are cyclic peptide molecules that also contain a fatty acid group. These five main classes of Fengycidn- and Dehydroxyfengycin-type metabolites are referred to as A, B, C, D and S. The backbone structure of these metabolites as well as the specific amino acid sequence for each of the five classes is shown in FIG. 1.

To determine the composition of the Fengycin- and Dehydroxyfengycin-type metabolites produced by Bacillus licheniformis strain CH200, these molecules were analyzed using UHPLC-TOF MS. Specifically, the molecular weights of the Fengycin-type metabolites produced by the CH200 strain after both 3 and 6 days growth in rich media (either in 869 or in M2 medium) at 30° C. were compared to the theoretical molecular weights expected for the Fengycin- and Dehydroxyfengycin-type metabolites. In addition, to determine the amino acid composition of these metabolites produced by the CH200 strain, peptide sequencing using LC-MS-MS was performed on each of the Fengycin-type metabolites identified via UHPLC-TOF MS.

In the manner described above, it was determined that Bacillus licheniformis strain CH200 produces Fengycins B, C, D, and S, but not Fengycin A, and produces all of the Dehydroxyfengycins A, B, C, D, and S.

In addition, it was further determined that the Bacillus licheniformis strain CH200 produces an additional class of Dehydroxyfengycin. In this class, the L-isoleucine of Dehydroxyfengycin B (position X₃ in FIG. 1) is replaced by L-homo-cysteine (Hcy). This Dehydroxyfengycin metabolite is referred to herein as Dehydroxyfengycin H and is shown in FIG. 1 and in Table II.

It was further determined that the Bacillus licheniformis strain CH200 produces an additional class of Fengycin and Dehydroxyfengycin. In this class, the amino acid at position 4 of the cyclic peptide backbone structure (position X₁ in FIG. 1) is replaced by L-isoleucine. These Fengycin and Dehydroxyfengycin metabolites are referred to herein as Fengycin I and Dehydroxyfengycin I and are shown in FIG. 1 and in Table II.

It was further determined that the CH200 strain produces derivatives of some of these compounds where the L-isoleucine at position 8 of the cyclic peptide chain (referred to as X₃ in FIG. 1) is replaced by L-methionine. These classes of Fengycin and Dehydroxyfengycin are referred to as MA, MB and MC, referring to derivatives of classes A, B and C in which the L-isoleucine at X₃ in FIG. 1 has been replaced by L-methionine. It was determined that Bacillus licheniformis strain CH200 produces Fengycin MA, but not Fengycin MB or MC and Dehydroxyfengycin MB and MC but not Dehydroxyfengycin MA. Theses molecules are also shown in FIG. 1 and in Table II.

Further, it was determined that the Bacillus licheniformis strain CH200 produces derivatives of these compounds in which the Tyrosine (Tyr) of Fengycin MB and Dehydroxyfengycin MB (position X₄ in FIG. 1) is replaced by the α-amino acid, Citruline. This class of Fengycin and Dehydroxyfengycin is referred to herein as Fengycin MB-Cit and Dehydroxyfengycin MB-Cit and is shown in FIG. 1 and in Table II.

In summary, these data demonstrate that the Bacillus licheniformis CH200 strain produces a very wide range of fengycin and dehydroxyfengycin molecules, which correlates well with the strong antifungal activity observed for this strain against the 13 plant fungal pathogens tested.

TABLE II
Summary of MS/MS identification of Fengycin- and Dehydroxyfengycin-
type metabolites in Bacillus licheniformis CH200 isolate

| | | | | | | Ring | Theoretical C16 | Theoretical | Observed |
|---|---|---|---|---|---|---|---|---|---|
| Homolog | X₁ | X₂ | X₃ | X₄ | R | Mass | Molecular Formula | C16 [M + H]⁺ | CH200 |
| Fengycin A | Ala | Thr | Ile | Tyr | OH | 1080.6 | C₇₂H₁₁₀N₁₂O₂₀ | 1463.8 | Not |
| | | | | | | | | | observed |
| Fengycin B | Val | Thr | Ile | Tyr | OH | 1108.7 | C₇₄H₁₁₄N₁₂O₂₀ | 1491.8 | C15, C16 |
| Fengycin C | Aba | Thr | Ile | Tyr | OH | 1094.6 | C₇₃H₁₁₂N₁₂O₂₀ | 1477.8 | C16, C17 |
| Fengycin D | Val | Thr | Val | Tyr | OH | 1094.6 | C₇₃H₁₁₂N₁₂O₂₀ | 1477.8 | C16, C17 |
| Fengycin S | Val | Ser | Ile | Tyr | OH | 1094.6 | C₇₃H₁₁₂N₁₂O₂₀ | 1477.8 | C16, C17 |
| Fengycin H | Val | Thr | Hcy | Tyr | OH | 1112.7 | C₇₂H₁₁₀N₁₂O₂₀S | 1495.8 | Not |
| | | | | | | | | | observed |
| Fengycin I | Ile | Thr | Ile | Tyr | OH | 1122.8 | C₇₇H₁₁₆N₁₂O₂₀ | 1505.8 | C16 |
| Fengycin MA | Ala | Thr | Met | Tyr | OH | 1098.7 | C₇₁H₁₀₈N₁₂O₂₀S | 1481.8 | C14 |
| Fengycin MB | Val | Thr | Met | Tyr | OH | 1126.8 | C₇₃H₁₁₂N₁₂O₂₀S | 1509.8 | Not |
| | | | | | | | | | observed |
| Fengycin MC | Aba | Thr | Met | Tyr | OH | 1112.7 | C₇₂H₁₁₀N₁₂O₂₀S | 1495.8 | Not |
| | | | | | | | | | observed |
| Dehydroxyfengycin A | Ala | Thr | Ile | Tyr | H | 1080.6 | C₇₂H₁₁₀N₁₂O₁₉ | 1447.8 | C14, C17 |
| Dehydroxyfengycin B | Val | Thr | Ile | Tyr | H | 1108.7 | C₇₄H₁₁₄N₁₂O₁₉ | 1475.8 | C15 |
| Dehydroxyfengycin C | Aba | Thr | Ile | Tyr | H | 1094.6 | C₇₃H₁₁₂N₁₂O₁₉ | 1461.8 | C16 |
| Dehydroxyfengycin D | Val | Thr | Val | Tyr | H | 1094.6 | C₇₃H₁₁₂N₁₂O₁₉ | 1461.8 | C16 |
| Dehydroxyfengycin S | Val | Ser | Ile | Tyr | H | 1094.6 | C₇₃H₁₁₂N₁₂O₁₉ | 1461.8 | C16 |
| Dehydroxyfengycin H | Val | Thr | Hcy | Tyr | H | 1112.7 | C₇₂H₁₁₀N₁₂O₁₉S | 1479.8 | C17 |
| Dehydroxyfengycin I | Ile | Thr | Ile | Tyr | H | 1122.8 | C₇₅H₁₁₆N₁₂O₁₉ | 1489.9 | C17 |
| Dehydroxyfengycin | Ala | Thr | Met | Tyr | H | 1098.7 | C₇₁H₁₀₈N₁₂O₁₉S | 1465.8 | Not |
| MA | | | | | | | | | observed |
| Dehydroxyfengycin | Val | Thr | Met | Tyr | H | 1126.8 | C₇₃H₁₁₂N₁₂O₁₉S | 1493.8 | C16 |
| MB | | | | | | | | | |
| Dehydroxyfengycin | Aba | Thr | Met | Tyr | H | 1112.7 | C₇₂H₁₁₀N₁₂O₁₉S | 1479.8 | C17 |
| MC | | | | | | | | | |
| Fengycin MB-Cit | Val | Thr | Met | Cit | OH | 1120.6 | C₇₀H₁₁₄N₁₄O₂₀S | 1503.8 | C16, C17 |
| Dehydroxyfengycin | Val | Thr | Met | Cit | H | 1120.6 | C₇₀H₁₁₄N₁₄O₁₉S | 1487.8 | C15, C16, |
| MB-Cit | | | | | | | | | C17 |

To identify secondary metabolite biosynthesis dusters found in CH201, a multi-step approach was used. First sequence data were searched using the antiSMASH analytical software platform to detect and putatively identify biosynthetic dusters responsible for the production of antagonistic compounds. These compounds can include ribosomally and non-ribosomally synthesized peptides, as well as other antagonistic compounds such as terpenes and siderophores. Once gene dusters of interest are putatively identified, they are subjected to a second round of BLASTp analysis to more definitively identify the cluster they belong to. This second round of BLASTp is necessary due to the fragmented nature of the whole genome sequence data, leading to biosynthetic dusters being broken up across multiple DNA fragments, as well as a final check on the identity of the cluster. LC-MS/MS analysis is, where possible, used to confirm the presence of specific secondary metabolites produced by the strain. In case bacteriocin-type synthesis pathways are putatively identified by antiSMASH the software package BAGEL, which is designed to identify small open reading frames that could encode antimicrobial peptides, is used to confirm them.

Lipopeptides—AntiSMASH analysis of the CH201 genome sequence resulted in the initial identification of biosynthetic dusters for two non-ribosomally synthesized lipopeptides, putatively a surfactin and a fengycin. In-depth BLASTp of the biosynthetic genes in these dusters identified the surfactin cluster and confirmed an incomplete fengycin-type gene cluster. AntiSMASH also identified another NRPS cluster, which BLASTp identified as bacilysin-type duster. Surfactins have been shown to show both antimicrobial activity and help colonization of the producing bacteria. Fengycins have been shown to have antagonistic effects against fungi, and to help prime the ISR and SAR responses in various plants, such as bean and tomato. Bacilysin has been demonstrated to have antibacterial as well as limited antifungal activities. The synthesis of these molecules is likely underlying the observed antagonistic properties of Bacillus subtilis CH201 against phytopathogenic fungi, including Aspergillus flavus, Fusarium graminearum, Fusarium oxysporum and Rhizoctonia solani.

Bacteriocins—AntiSMASH also identified a lantipeptide biosynthesis cluster in Bacillus subtilis CH201, which was confirmed by BAGEL to be a subtilosin A biosynthesis cluster. Subtilosin A has been to shown to have activity against other Gram positive bacteria.

Others—AntiSMASH Identified terpene biosynthesis genes in Bacillus subtilis CH201 which could function as both antagonistic molecules or as signaling molecules between the bacteria and the host plant. Furthermore, a siderophore biosynthesis cluster was found for Bacillibactin. Bacteria have developed several distinct mechanisms to compete for iron, an element whose availability often limits microbial growth. These include specific iron uptake transporters, the secretion of large numbers of diverse siderophores, and the synthesis of siderophore receptors to utilize heterologously produced siderophores from other microorganisms. Siderophores are known to have an antagonistic effect by depriving iron from other microorganisms. The presence of an efficient iron uptake system can therefore contribute to protect the host plant against pathogenic infections.

Secondary metabolite production—Based on genome analysis, various lipopeptides, bacteriocins and other secondary metabolites were predicted to be synthesized by Bacillus subtilis CH0201. UPLC-MS analysis was used to determine the spectrum of secondary metabolites produced by Bacillus subtilis CH201. Subsequently, the secondary metabolite profile of Bacillus subtilis CH201, when grown for six days in rich medium, was compared to the predicted metabolome. Based on the genome sequence of B. subtilis CH201, biosynthetic pathways for the production of several key metabolites were predicted; however, only surfactin-type compounds were identified by LC-MS in the culture supernatant of B. subtilis CH201 (Table II). Although a fengycin-type gene duster was identified by antISMASH, none of fengycin-type compounds were detected, confirming that the incomplete gene cluster is not functional. The detection and identification of the metabolites by mass spectrometer could be hampered by various factors, such as culture conditions, chromatographic conditions, different ionization pattern of each molecule, etc. Furthermore, the synthesis of the secondary metabolites is dependent on many environmental factors, such as growth medium, pH, temperature, oxygenation, or the lack of ferric iron (for siderophore biosynthesis). However, it should be noted that under the same culture conditions, a Bacillus subtilis strain used as a positive control showed a significantly more diverse metabolic profile compared to the CH201, including the synthesis of fengycins and subtilosin, this besides surfactins. The results of the LC-MS analysis of B. subtilis CH201 are presented in Table III.

TABLE III
Predicted gene clusters for secondary metabolite synthesis and their confirmation by UPLC-MS analysis

| | FA | | chemical | | gene cluster | mass detected by |
|---|---|---|---|---|---|---|
| compound | chain | [M + H]⁺ | formula | Target | (antiSMASH 3.0) | UPLC-MS |
| Bacillibactin | N.A. | 883.2628 | C39H42N6O18 | iron chelation | 1 complete cluster: 99.8% ID | absent |
| | | | | | present | |
| Bacilysin | N.A. | 271.1288 | C12H18N2O5 | Gram + | 1 complete cluster: 99.8% ID | absent |
| | | | | Staphylococci | present | |
| Subtilosin | N.A. | 3400.5798 | C152H226N38O45S3 | closely related | 2 genes (precursor and | absent |
| | | | | species | biosynthesis: 100% ID) | |
| | | | | | present | |
| Surfactin | C11 | 980.6278 | C49H85N7O13 | antibacterial | (1 complete cluster: 99.5% ID) | absent |
| | | | | | present | |
| Surfactin | C12 | 994.6434 | C50H87N7O13 | antibacterial | (1 complete cluster: 99.5% ID) | present |
| | | | | | present | |
| Surfactin | C16 | 1050.706 | C54H95N7O13 | antibacterial | (1 complete cluster: 99.5% ID) | present |
| | | | | | present | |
| Surfactin A | C13 | 1008.6591 | C51H89N7O13 | antibacterial | (1 complete cluster: 99.5% ID) | present |
| | | | | | present | |
| Surfactin B | C14 | 1022.6747 | C52H91N7O13 | antibacterial | (1 complete cluster: 99.5% ID) | present |
| | | | | | present | |
| Surfactin C | C15 | 1036.6904 | C53H93N7O13 | antibacterial | (1 complete cluster: 99.5% ID) | present |
| | | | | | present | |
| Surfactin C | C17 | 1064.7217 | C55H97N7O13 | antibacterial | (1 complete cluster: 99.5% ID) | present |
| | | | | | present | |

Based on genome analysis, various lipopeptides, bacteriodns and other secondary metabolites were predicted to be synthesized by Bacillus subtilis CH201. These molecules, their lipid modification, predicted molecular mass, chemical formula, known function based on literature, and their presence or absence in the culture supernatant of Bacillus subtilis CH201 grown for 6 days in 869 or M2 medium are presented. NA. for fatty acid (FA) chain: no lipid modification.

### Example 2

### Seed Treatment with a Combination of Bacillus Licheniformis CH200 and Bacillus Subtilis CH201 Reduces Disease and Increases Yield in Soybean and Corn

### Formulations

Various formulations were produced for seed treatment testing by homogenizing the listed Ingredients in powder form. Simple mixing of the Ingredients by homogenization was preferable to any form of grinding. Three formulations are shown below (Formulations T1, T2, and T3).

### Formulation T1:

| | | | Concentration | Concentration |
|---|---|---|---|---|
| Raw-Materials | CAS Number | Function | (% p/p) | (g/Kg) |
| Bacillus licheniformis CH200 | — | Active Ingredient | 20.00 | 200.00⁽¹⁾ |
| Bacillus subtilis CH201 | — | Active Ingredient | 20.00 | 200.00⁽²⁾ |
| Maltodextrin | 9050-36-6 | Vehicle | 20.00 | 200.00 |
| Silica | 112945-52-5 | Vehicle | 10.00 | 100.00 |
| Calcium carbonate | 1317-65-3 | Vehicle | 30.00 | 300.00 |

| | | |
|---|---|---|
| Total | 100.00 | 1000.00 |

### Formulation T2:

| | | | Concentration | Concentration |
|---|---|---|---|---|
| Raw-Materials | CAS Number | Function | (% p/p) | (g/Kg) |
| Bacillus licheniformis CH200 | — | Active Ingredient | 20.00 | 200.00⁽¹⁾ |
| Bacillus subtilis CH201 | — | Active Ingredient | 20.00 | 200.00⁽²⁾ |
| Maltodextrin | 9050-36-6 | Vehicle | 10.00 | 100.00 |
| Silica | 112945-52-5 | Vehicle | 10.00 | 100.00 |
| Calcium carbonate | 1317-65-3 | Vehicle | 40.00 | 400.00 |

| | | |
|---|---|---|
| Total | 100.00 | 1000.00 |

### Formulation T3:

| | | | Concentration | Concentration |
|---|---|---|---|---|
| Raw-Materials | CAS Number | Function | (% p/p) | (g/Kg) |
| Bacillus licheniformis CH200 | — | Active Ingredient | 20.00 | 200.00⁽¹⁾ |
| Bacillus subtilis CH201 | — | Active Ingredient | 20.00 | 200.00⁽²⁾ |
| Silica | 112945-52-5 | Vehicle | 10.00 | 100.00 |
| Calcium carbonate | 1317-65-3 | Vehicle | 50.00 | 500.00 |

| | | |
|---|---|---|
| Total | 100.00 | 1000.00 |

The manufacture of these compositions includes addition of the following Ingredients in powder form in the order specified: 1) maltodextrin; 2) calcium carbonate; 3) silicon dioxide; 4) CH1200; and 5) CH201, with a homogenization time of about 5 min.

Additional wettable powder formulations were prepared similarly.

### Formulation T4:

| | Technical | Formulation |
|---|---|---|
| Ingredients | cfu/g | Nominal weight % |
| CH200, Bacillus Licheniformis | 8.60 × 10¹¹ | 20 |
| CH201, Bacillus Subtilis | 8.10 × 10¹¹ | 20 |
| maltodextrin | | 40 |
| Calcium carbonate | | 16 |
| silica | | 4 |

### Formulation T5:

| | Technical | Formulation |
|---|---|---|
| Ingredients | cfu/g | Nominal weight % |
| CH200, Bacillus Licheniformis | 8.60 × 10¹¹ | 6.5 |
| CH201, Bacillus Subtilis | 8.10 × 10¹¹ | 6.5 |
| maltodextrin | | 86 |
| silica | | 1 |

A suspension concentrate was also prepared as Formulation SC1. The ingredients summarized below were mixed as described below to prepare a suspension concentrate.

| | | Formulation | | |
|---|---|---|---|---|
| | Technical | | Nominal | cfu/g, ml |
| Ingredients | cfu/g | Weight (g) | weight % | cfu/g |
| CH200, Bacillus | 8.60 × 10¹¹ | 29.5 | 2.95 | 2.53 × 10¹⁰ |
| Licheniformis | | | | |
| CH201, Bacillus | 8.10 × 10¹¹ | 31.3 | 3.13 | 2.53 × 10¹⁰ |
| Subtilis | | | | |
| Potassium sorbate | | 1.0 | 0.10 | |
| Ascorbic acid | | 4.0 | 0.40 | |
| Modified sodium | | 15.0 | 1.50 | |
| lignosulfate | | | | |
| Sodium | | 11.0 | 1.10 | |
| tripolyphosphate | | | | |
| Urea | | 18.0 | 1.80 | |
| Sodium bisulfate | | 24.0 | 2.40 | |
| Glycerin | | 350.0 | 35.00 | |
| Dow AF | | 0.5 | 0.05 | |
| (xiameter) | | | | |
| antifoam | | | | |
| Citric acid | | 2.3 | 0.23 | |
| Na₃Citrate | | 0.1 | 0.01 | |
| Xanthan gum | | 0.2 | 0.02 | |
| Water | | 513.2 | 51.32 | |
| Total | | 1000 | 100 | |

A vessel equipped with an agitator was charged with the calculated amount of water to provide 1 kg of total formulation. Potassium sorbate, modified sodium lignosulfate and sodium tripolyphosphate were added to the vessel, and the mixture was agitated until all the salts were dissolved. Urea, ascorbic add and sodium bisulfate were added, and the mixture was agitated until all ingredients were dissolved. The antifoam (xiameter) was added, followed by addition of FMCH001 and FMCH002 under vigorous agitation. The slurry was further homogenized until a particle size d90 of less than 20 um, preferably less than 10 um, was achieved. The pH of the slurry was adjusted with sodium citrate and citric acid, until pH=4.0. Finally, glycerin and xanthan gum were added under agitation to produce the formulation as a slurry.

Additional SC formulations were prepared similarly, as described below.

| | | Formulation | | |
|---|---|---|---|---|
| SC2 | Technical | | Nominal | cfu/g, ml |
| Ingredients | cfu/g | Weight (g) | weight % | cfu/g |
| CH200, Bacillus | 4.60 × 10¹¹ | 15.1 | 5.09 | 2.34 × 10¹⁰ |
| Licheniformis | | | | |
| CH201, Bacillus | 4.90 × 10¹¹ | 14.20 | 4.79 | 2.35 × 10¹⁰ |
| Subtilis | | | | |
| Potassium sorbate | | 1.0 | 0.3 | |
| Ascorbic acid | | 1.0 | 0.3 | |
| Sodium | | 4.0 | 1.3 | |
| lignosulfate | | | | |
| Sodium | | 10.0 | 3.4 | |
| polytriphosphate | | | | |
| Urea | | 2.0 | 0.7 | |
| Citric acid | | 5.0 | 1.7 | |
| Sucrose | | 120.0 | 40.5 | |
| Dow AF | | 0.3 | 0.1 | |
| (xiameter) | | | | |
| antifoam | | | | |
| Water | | 124.0 | 41.8 | |
| Total | | 296.6 | 100 | |

| | | Formulation | | |
|---|---|---|---|---|
| SC3 | Technical | | Nominal | cfu/g, ml |
| Ingredients | cfu/g | Weight (g) | weight % | cfu/g |
| CH200, Bacillus | 8.2 × 10¹¹ | 10.0 | 3.65 | 2.99 × 10¹⁰ |
| Licheniformis | | | | |
| CH201, Bacillus | 4.70 × 10¹¹ | 17.4 | 6.35 | 2.99 × 10¹⁰ |
| Subtilis | | | | |
| Potassium | | 5.0 | 1.8 | |
| phosphate | | | | |
| Potassium sorbate | | 1.0 | 0.4 | |
| Ascorbic acid | | 0.5 | 0.2 | |
| Sodium | | 5.0 | 1.7 | |
| lignosulfate | | | | |
| Potassium | | 5.0 | 1.8 | |
| phosphate | | | | |
| Sodium | | 25.0 | 9.1 | |
| hexameta- | | | | |
| phosphate | | | | |
| Urea | | 50.0 | 18.2 | |
| Yeast extract | | 5.0 | 1.8 | |
| Glycerin | | 55.0 | 20.1 | |
| Dow AF | | 0.1 | 0.04 | |
| (xiameter) | | | | |
| antifoam | | | | |
| Water | | 100 | 36.5 | |
| Total | | 274.0 | 100 | |

| | | Formulation | | |
|---|---|---|---|---|
| SC4 | Technical | | Nominal | cfu/g, ml |
| Ingredients | cfu/g | Weight (g) | weight % | cfu/g |
| CH200, Bacillus | 8.2 × 10¹¹ | 10.0 | 3.65 | 3.00 × 10¹⁰ |
| Licheniformis | | | | |
| CH201, Bacillus | 4.70 × 10¹¹ | 17.48 | 6.38 | 3.00 × 10¹⁰ |
| Subtilis | | | | |
| Potassium | | 1.0 | 0.4 | |
| sorbate | | | | |
| Ascorbic acid | | 0.5 | 0.2 | |
| Sodium | | 5.0 | 1.9 | |
| lignosulfate | | | | |
| Potassium | | 5.0 | 1.8 | |
| phosphate | | | | |
| Sodium hexa- | | 25.0 | 9.1 | |
| metaphosphate | | | | |
| Urea | | 50.0 | 18.2 | |
| Fertilizer | | 10.0 | 3.6 | |
| Glycerin | | 55.0 | 20.1 | |
| Dow AF | | 0.1 | 0.04 | |
| (xiameter) | | | | |
| antifoam | | | | |
| Water | | 95 | 34.7 | |
| Total | | 274.1 | 100 | |

| | | Formulation | | |
|---|---|---|---|---|
| SC5 | Technical | | Nominal | cfu/g, ml |
| Ingredients | cfu/g | Weight (g) | weight % | cfu/g |
| CH200, Bacillus | 8.2 × 10¹¹ | 9.2 | 3.66 | 3.00 × 10¹⁰ |
| Licheniformis | | | | |
| CH201, Bacillus | 4.70 × 10¹¹ | 16.1 | 6.4 | 3.00 × 10¹⁰ |
| Subtilis | | | | |
| Potassium | | 1.0 | 0.4 | |
| sorbate | | | | |
| Ascorbic acid | | 1.0 | 0.4 | |
| Sodium | | 4.2 | 1.7 | |
| lignosulfate | | | | |
| Ethylene Oxide/ | | 1.0 | 0.4 | |
| Propylene Oxide | | | | |
| Block Copolymer | | | | |
| Citric acid-H₂0 | | 2.0 | 0.8 | |
| Sodium citrate | | 2.0 | 0.8 | |
| Attapulgite | | 4.75 | 1.9% | |
| Yeast extract | | 3.00 | 1.2% | |
| Glycerin | | 34.00 | 13.5% | |
| Water | | 68.0 | 27.0 | |
| Total | | 251.6 | 100 | |

| | | Formulation | | |
|---|---|---|---|---|
| SC6 | Technical | | Nominal | cfu/g, ml |
| Ingredients | cfu/g | Weight (g) | weight % | cfu/g |
| CH200, Bacillus | 6.00 × 10¹¹ | 97.49 | 4.35 | 2.61 × 10¹⁰ |
| Licheniformis | | | | |
| CH201, Bacillus | 3.00 × 10¹¹ | 219.24 | 9.78 | 2.93 × 10¹⁰ |
| Subtilis | | | | |
| Potassium | | 7.5 | 0.3 | |
| sorbate | | | | |
| Ascorbic acid | | 7.5 | 0.33 | |
| Sodium | | 30 | 1.3 | |
| lignosulfate | | | | |
| Sodium | | 22.5 | 1.0 | |
| polytriphosphate | | | | |
| Sodium bisulfate | | 66.0 | 2.9 | |
| Urea | | 15.0 | 0.7% | |
| Dow AF | | 2.25 | 0.1% | |
| (xiameter) | | | | |
| Glycerin | | 675.00 | 30.1% | |
| Water | | 1100.0 | 49.1 | |
| Total | | 2242.5 | 100 | |

A Wettable Powder formulation was prepared by mixing the components described below.

| | | Formulation | | |
|---|---|---|---|---|
| WP1 | Technical | | Nominal | cfu/g, ml |
| Ingredients | cfu/g | Weight (g) | weight % | cfu/g |
| CH200, Bacillus | 8.2 × 10¹¹ | 4.6 | 3.67 | 3.01 × 10¹⁰ |
| Licheniformis | | | | |
| CH201, Bacillus | 4.7 × 10¹¹ | 8.0 | 6.39 | 3.00 × 10¹⁰ |
| Subtilis | | | | |
| Yeast extract | | 10.0 | 8.0 | |
| Silica | | 5.0 | 4.0 | |
| Sodium lignosulfate | | 9 | 7.2 | |
| lignosulfate | | | | |
| Monopotassium | | 12.0 | 9.6 | |
| phosphate | | | | |
| Sodium hexa- | | 66.0 | 2.9 | |
| metaphosphate | | | | |
| Urea | | 14.8 | 11.8 | |

| | | |
|---|---|---|
| Total | 129.4 | 100 |

### Field Trial Protocols

A formulation was produced with active ingredients of a 1:1 ratio of spores of Bacillus licheniformis CH200 and spores of Bacillus subtilis CH201 at a concentration of 2.5×10¹¹ CFU/ml.

Field trials of corn and soybean were conducted by applying this formulation at seed planting along with untreated seed and seeds treated with commonly utilized chemical active ingredient fungicides/Insecticides, shown below as AI1, AI2, AI3, AI4, and AI5. The chemical active Ingredient compositions were applied according to known effective rates. The rate of application of AI5 is detailed below. The CFU/seed of Bacillus licheniformis CH200 and Bacillus subtilis CH201 used in the field trials is designated in Tables IV-VI below, which show the results of the soybean and corn trials. The field trials were performed under fungal disease pressure.

Chemical active Ingredient composition 1 (AI1): Thiamethoxam at 22.61%, Mefenoxamat 1.70%, Fludioxonil at 1.12%, and other Ingredients at 74.57%.

Chemical active Ingredient composition 2 (AI2): Fludioxonil at 40.3% and other Ingredients at 59.7%.

Chemical active Ingredient composition 3 (AI3): Metalaxyl: N-(2,6-dimethylphenyl)-N-(methoxyacetyl)alaninemethylester at 28.35% and other Ingredients at 71.65%.

Chemical active Ingredient composition 4 (AI4): Clothianidin at 48.0% and other Ingredients at 52.0%.

Chemical active Ingredient composition 5 (AI5): Fludioxonil, Mefenoxam, Thiophanate-methyl, and Thiamethoxam applied at a rate on seed of 2.5-, 7.5-, 10-, and 50-g ai/100 kg, respectively.

TABLE IV
Results of soybeans field trial under Rhizoctonia disease pressure with
seed treated with chemical active ingredients Al1 and a combination of a 1:1 ratio
of Bacillus licheniformis CH200 and Bacillus subtilis CH201

| | | | Disease | | | Disease | | |
|---|---|---|---|---|---|---|---|---|
| | | % Emerge | Level | % Emerge | Vigor | Level | Vigor | Yield |
| Treatment | | 10 DAP | 10 DAP | 24 DAP | 24 DAP | 24 DAP | 38 DAP | bu/Acre |
| 1 | Untreated | 39.2 | 2.0 | 57.7 | 6.0 | 2.0 | 6.3 | 66.6 |
| | Seed | | | | | | | |
| 2 | Al1 | 53.5 | 1.9 | 65.4 | 6.5 | 1.8 | 6.8 | 70.6 |
| 3 | Al1 + 1:1 ratio | 59.1 | 1.6 | 68.7 | 7.3 | 1.4 | 7.3 | 72.1 |
| | CH200, CH201 | | | | | | | |
| | (5 × 10⁵ | | | | | | | |
| | CFU/seed) | | | | | | | |

As apparent from Table IV above, a significant improvement was observed for each of percent emergence, disease level, and vigor for all time points with the application of the combination of the CH200 and CH201 strains as compared to AI1 chemical actives alone. In addition, yield is increased by approximately 1.5 bu/acre with application of the combination of the CH200 and CH201 strains as compared to AI1 chemical actives alone.

TABLE V
Results of soybeans field trial under high Rhizoctonia disease pressure with seed
treated with chemical active ingredients Al5 and varying amounts of a combination of a
1:1 ratio of Bacillus licheniformis CH200 and Bacillus subtilis CH201

| | | Disease | | | Disease | |
|---|---|---|---|---|---|---|
| | % Emerge | Level | % Emerge | Vigor | Level | Yield |
| Treatment | 11 DAP | 11 DAP | 25 DAP | 25 DAP | 25 DAP | bu/Acre |
| Untreated Seed | 41.7 | 2.0 | 62.5 | 6.0 | 2.0 | 71.1 |
| Al5 | 59.2 | 1.5 | 69.4 | 6.5 | 1.0 | 75.8 |
| Al5 + 1:1 CH200, CH201 | 65.0 | 1.0 | 70.2 | 7.5 | 0.8 | 78.4 |
| (5 × 10⁵ CFU/seed) | | | | | | |
| Al5 + 1:1 CH200, CH201 | 65.5 | 1.0 | 71.8 | 8.0 | 0.8 | 79.3 |
| (5 × 10⁶ CFU/seed) | | | | | | |

The test was performed at natural field conditions, with a high disease pressure.

As apparent from Table V above, a significant improvement was observed for each of percent emergence, disease level, and vigor for all time points with the application of both rates of the combination of the CH200 and CH201 strains (5×10⁵ CFU/seed and 5×10 CFU/seed) as compared to AI5 chemical actives alone. In addition, yield is increased by approximately 2.6 and 3.5 bu/acre with application of the combination of the CH200 and CH201 strains (5×10⁵ CFU/seed and 5×10 CFU/seed, respectively) as compared to AI5 chemical actives alone.

TABLE VI
Results of corn field trial under Rhizoctonia disease pressure with seed treated
with chemical active ingredients Al2 + Al3 + Al4 and varying amounts of a combination
of a 1:1 ratio of Bacillus licheniformis CH200 and Bacillus subtilis CH201

| | | | | | | Rhizoctonia |
|---|---|---|---|---|---|---|
| | Rhizoctonia | Rhizoctonia | Rhizoctonia | Rhizoctonia | Rhizoctonia | sp. |
| | sp. | sp. | sp. | sp. | sp. | Grain yield |
| | % Emerge | % Emerge | Vigor | Diseased | % Emerge | Bu/A |
| | 14 DAP | 30 DAP | 30 DAP | 30 DAP | 39 DAP | 161 DAP |
| Untreated Seed | 63 | 70 | 6.0 | 2.0 | 70 | 228.7 |
| CC | 89 | 89 | 7.5 | 1.5 | 89 | 229.7 |
| CC + 1:1 CH200, CH201 | 90 | 92 | 9.0 | 0.3 | 92 | 248.7 |
| (5 × 10⁵ CFU/seed) | | | | | | |
| CC + 1:1 CH200, CH201 | 91 | 92 | 9.5 | 0.3 | 92 | 253.9 |
| (5 × 10⁶ CFU/seed) | | | | | | |
| CC + 1:1 CH200, CH201 | 93 | 94 | 9.5 | 0.3 | 94 | 253.5 |
| (2.5 × 10⁷ CFU/seed) | | | | | | |

CC = Al2 + Al3 + Al4

As apparent from Table VI above, a significant improvement was observed for each of percent emergence, disease level, and vigor for all time points with the application of all 3 rates of the combination of the CH200 and CH201 strains (5×10⁵ CFU/seed, 5×10 CFU/seed, and 2,5×10⁷ CFU/seed) as compared to AI2+AI3+AI4 chemical actives alone. In addition, yield is increased by approximately 10% with application of the combination of the CH200 and CH201 strains as compared to use of the chemical actives alone.

### Example 3

### Seed Treatment with a Combination of Bacillus Licheniformis CH200 and Bacillus Subtilis CH201 Increases Yield in Soybean Artificially Inoculated with Rhizoctonia solani

Experiments were performed to investigate the effect on emergence, root disease, and yield in soybean in the presence of Rhizoctonia disease pressure when seeds were treated with a combination of the CH200 and CH201 strains in addition to chemical active agents for pathogen control.

Specifically, an experiment in soybean was set up as follows: 1) seed was untreated (UTC); 2) seed was treated with a combination of Fludioxonil+Mefenoxam+Thiophanate-Methyl (TPM)+Thiamethoxam at a rate of about 2.5-, 7.5-, 10, and 50-g/100 g seed, respectively, (referred to as “Chem Control”; 3) seed was treated with Chem Control plus a combination of 2×10⁺⁵ cf u/seed of each of strain CH200 and CH201; and 4) seed was treated with CRUISERMAXX® (insecticide plus fungicide, containing active ingredients thiamethoxam, fludioxonil plus metalaxyl-M; SYNGENTA CROP PROTECTION, INC) plus VIBRANCE® (active Ingredient Sedaxane; SYNGENTA CROP PROTECTION, INC) according to label instructions. Two trials were performed in Whitewater, Wis. with 4 replicates per treatment per trial. The trials were inoculated with Rhizoctonia solani by first growing the pathogen separately on moistened autoclaved grain seed and subsequently the dried inoculum was mixed with the seed at the time of planting to a prescribed rate to provide infection when the seed commenced to grow. The average soybean emergence, root disease scoring on a scale of 0-5 (0=no disease and 5=severe disease), and yield results for the trials are presented in Table VII below.

The results in Table VII show that treating with the combination of the Bacillus licheniformis CH200 and Bacillus subtilis CH201 in addition to the Chem Control resulted in improvements in percent emergence, level of root disease, and yield over that of the chemical active agent alone. In addition, the combination of CH200 and CH201 outperformed the commercial product CRUISERMAXX®+VIBRANCE® in both controlling root disease and in improved yield. Specifically, the combination of CH200 and CH201 in addition to the Chem Control resulted in an average increase in yield of 3.1 bushels per acre over that of the chemical active agent alone (from 51.4 to 54.5 bushels per acre). Thus, seed treatment with the combination of CH200 and CH201 can provide significant improvement in yields in soybean, even under conditions of severe pathogen pressure.

TABLE VII
Average results on soybean emergence, root disease, and yield in field
trials with plants artificially inoculated with Rhizoctonia solani with
treatment of the soybean seed with a combination of B. licheniformis
CH200 and B. subtilis CH200 in addition to chemical active agent
treatment for soybean seeds.

| | | | Root Disease | |
|---|---|---|---|---|
| | | % | Level¹ | Yield |
| Treatment | | Emergence | (0-5) | (Bu/Acre) |
| 1 | Untreated Seed | 60 | 2.0 | 45.4 |
| 2 | Chem Control | 64 | 1.5 | 51.4 |
| 3 | Chem Control + CH200, | 68 | 0.9 | 54.5 |
| | CH201 (4 × 10⁵ | | | |
| | CFU/seed) | | | |
| 4 | CRUISERMAXX ® + | 68 | 1.25 | 53.2 |
| | VIBRANCE ® | | | |

¹= no root rot,
⁵= severe root rot

### Example 4

### Bacillus licheniformis CH200 with Bacillus subtilis CH201 Reduces Fusarium Root Rot and Increases Yield in Corn

An experiment in corn was performed to determine the ability of the combination of the Bacillus licheniformis CH200 and Bacillus subtilis CH201 strains to prevent and reduce root rot caused by Fusarium graminearum. The experiment was set up as follows: 1) seed was untreated; 2) seed was treated with a combination of Fludioxonil (0.0625 mg/seed)+Mefenoxam (0.0625 mg/seed)+Clothianidin (0.25 mg/seed) referred to as “Chem Control”; and 3) seed was treated with Chem Control plus a combination of 2.5×10⁺⁶ cfu/seed of each of strain CH200 and CH201.

Field trials were performed in Whitewater Wis. on soil that was Inoculated with Fusarium graminearum, a causal agent of seed rot and seedling blignt. F. graminearum was grown on moisten autoclaved grain seed. After the grain seed was covered with mycelia growth, the seed was air dried and subsequently ground up. The prepared ground inoculum was planted along with the corn seed at the prescribed rate to ensure higher and more uniform infection rates. This disease can cause root discoloration, reduce emergence and reduce yields. The plant count, amount of root rot reduction, and yield of corn was determined for each treatment and the results are shown below in Table VIII.

Results in Table VIII below show that Inoculation of the seed with the combination of the Bacillus licheniformis CH200 and Bacillus subtilis CH201 strains increased yield of corn when compared to seeds that were treated with the Chem Control alone. Specifically, the combination of CH200 and CH201 in addition to the Chem Control resulted in an average increase in yield of 14.5 bushels per acre in three trials over that of the chemical active agents alone. In addition, emergence was increased with the application of CH200 and CH201 and levels of root rot caused by Fusarium reduced significantly from chemical base treatment with the later timing demonstrating the most improvement.

TABLE VIII
Yield results for corn after seed treatment with B. licheniformis CH200 and
B. subtilis CH201 in combination with chemical active agents in soil inoculated
with Fusarium graminearum, a causal agent of seed rot and root rot of corn.

| | | | Root Rot | Root Rot | |
|---|---|---|---|---|---|
| | 1st Plant | 2nd Plant | Reduction | Reduction | Yield |
| TREATMENT | Count | Count | 1st Timing % | 2nd Timing % | bu/acre |
| Untreated Seed | 65 | 71 | 0 | 0 | 158.1 |
| Chem Control | 83 | 88 | 35 | 30 | 212.4 |
| Chem Control + | 89 | 91 | 45 | 55 | 226.9 |
| CH200, CH201 | | | | | |
| (5 × 10⁶ CFU/seed) | | | | | |

### Example 5

### Impact of Biological Seed Treatments on Growth and Yield of Cotton Inoculated with Rhizoctonia

A field trial was conducted in Georgia to test the efficacy of the biological combination of the invention as a seed treatment of cottons Inoculated with Rhizoctonia species.

Cotton seeds were treated with separate slurries being prepared for the chemical and biological treatments which were simultaneously applied to the seed. Seeds were placed in a jar which was shaken on a modified paint shaker until the product was uniformly coated on the seeds. The base chemical treatment comprised three commercial formulations comprising 1) 40.3% of fludioxonil, 2) 33.3% of mefenoxam, and 3) 12.7% of imidacloprid, applied at 5.2+20.8+165 mL/100 kg respectively for 191 mL/100 kg of seeds of formulated products with the addition of colorant, polymer and water for a final slurry rate of 765 mL/100 kg. Another comparison chemical treatment included 10 mL/100 kg of seeds of a commercial formulation of 43.7% of sedaxane, in addition to the base chemical treatment. Other seeds were treated with the base chemical treatment+a suspension concentrate (SC1) of the biological formulation applied at 11 mL per 100,000 seeds.

The trial comprised randomized complete block tests using 6 foot by 30 foot (about 1.8 meters by 9.1 meters) plots. Treated and untreated cotton (Gossypium hirsutum, var PHY400) seeds were planted. Using a cone planter, seeds were planted 3 seeds/ft, 1 inch deep with row spacing of 36 Inches in sandy loam soil for a total of 180 seeds per plot. The plots were inoculated with Rhizoctonia sp. to ensure infestation. The plots and cotton plants were treated under generally accepted agronomic conditions for cotton including conventional tillage and irrigation as needed until they were ready for harvest. The plants were assessed during the growing period for emergence, vigor (on a 1-5 qualitative scale), phytotoxicity, and then harvested to assess yield. The results are summarized in Table IX.

TABLE IX

| | Rating | Plant Evaluation | | | Base | Base + | Base + |
|---|---|---|---|---|---|---|---|
| Rating | Unit | Interval | | Untreated | Chemical | SC1 | sedaxane |
| Emergence | % | 9 | DAP | 23.1 | 44.6 | 68.6 | 63.1 |
| | | 16 | DAP | 22.5 | 43.9 | 71 | 63.3 |
| | | 23 | DAP | 24.4 | 40 | 71.7 | 62.6 |
| | | 30 | DAP | 21.1 | 39.4 | 72.8 | 63.6 |
| Vigor | 1-10 | 9 | DAP | 5 | 7 | 9 | 8 |
| | | 16 | DAP | 4.3 | 6.8 | 8.8 | 8.8 |
| | | 23 | DAP | 4 | 5.3 | 8.5 | 9 |
| | | 30 | DAP | 5.5 | 6.5 | 9 | 8.8 |
| Yield | Lb/Acre | 184 | DAP | 2390 | 2942 | 4026 | 3826 |
| | Kg/Ha | | | 2677 | 3295 | 4509 | 4285 |

All seed treatments had a higher stand than the untreated control at all 4 assessment timings. At all four assessment timings, the biological treatment provided the best emergence. The untreated seed had the lowest vigor at all 4 assessment timings. The biological treatment provided stronger vigor than the untreated control and the base treatment, and about equal to the base+sedaxane treatment. All products were significantly higher in yield than the UTC. The biological treatment outperformed both chemical treatments.

### Example 6

### Impact of Biological Seed Treatments on Growth and Yield of Peanuts Inoculated with Rhizoctonia

A field trial was conducted in Georgia to test the efficacy of the biological combination of the invention as a seed treatment for peanuts Inoculated with Rhizoctonia species.

The trial comprised randomized complete block tests using 6 foot by 30 foot (about 1.8 meters by 9.1 meters) plots. Treated and untreated peanut (Arachis hypogaes, var GA06G) seeds were planted. Seeds were treated with a base chemical treatment comprising a commercial formulation of 3.2% of azoxystrobin, 2.0% of fludioxonil and 0.4% of mefenoxam (94.4 other Ingredients) and the base chemical treatment plus the biological formulation. Using a cone planter, seeds were planted 1.25 Inches deep with row spacing of 36 Inches in sandy loam soil. The plots were inoculated with Rhizoctonia sp. to ensure infestation. The plots and peanut plants were treated under generally accepted agronomic conditions for peanuts including conventional tillage and irrigation as needed until they were ready for harvest. The plants were assessed during the growing period for emergence, vigor (on a 1-5 qualitative scale), phytotoxicity, and then harvested to assess yield. The results are summarized in Table X.

TABLE X

| | | Plant | | | |
|---|---|---|---|---|---|
| | | Evaluation | Untreated | | Base + |
| Description | Unit | Interval | Seed | Base | T4 |
| Seed | g Al/100 kg | | 0 | 195 | 195 + 17 |
| Treatment | seed | | | | |
| Emergence | % | 8 DAP | 2.6 | 10.3 | 13.6 |
| | | 11 DAP | 11 | 27.2 | 38.6 |
| | | | 15 | 41.4 | 42.2 |
| Vigor | 1-5 | 8 DAP | 1.8 | 3 | 4.5 |
| | | 11 DAP | 2.3 | 2.8 | 4.3 |
| | | | 1.8 | 2.5 | 4.8 |
| Phytotoxicity | % | 8 DAP | 0 | 0 | 0 |
| | | 11 DAP | 0 | 0 | 0 |
| Yield | lb/acre | | 1276.6 | 3230.7 | 3660.3 |
| | kg/Ha | | 1429.8 | 3618.4 | 4099.5 |

No phytotoxicity was observed for any seed treatment. The seed treatments were significantly improved compared to the untreated seed at all three assessment timings for plant counts. At the first count and second count, the biological+base treatment was improved over the base chemical treatment. At the third assessment timing, the biological seed treatments were similar to the base treatment. These results show that the biological treatment provided faster emergence of plants compared to the chemical treatment alone.

The untreated seeds had the lowest vigor at all 3 assessment timings with the biological seed treatment being significantly improved over the untreated seeds. The biological treatment also had improved vigor over the chemical treatment at all three assessments.

Yield at harvest for the biological treatment was significantly improved over the untreated and chemical treatment plots.

1. A method for controlling plant fungal pathogens, the method comprising:
delivering to seed of a plant, roots of a plant, or soil surrounding a plant a composition comprising:
a biologically pure culture of a Bacillus licheniformis CH200 deposited as DSM 17236, or a mutant thereof having all the identifying characteristics thereof; and
a biologically pure culture of a Bacillus subtilis CH201 deposited as DSM 17231, or a mutant thereof having all the identifying characteristics thereof,
wherein delivery of the composition improves plant yield in the presence of a plant fungal pathogen, improves plant resistance to a plant fungal pathogen, or reduces plant infection by a fungal pathogen, and combinations thereof.

2. The method of claim 1, wherein the plant fungal pathogen comprises one or more of a Botrytis spp., a Botrytis cinerea, a Fusarium spp., a Fusarium colmorum, a Fusarium oxysporum, a Phytophthora spp., a Phytophthora capsid, a Rhizoctonia spp., a Rhizoctonia solani, a Magnaporthe oryzae, a Pythium spp., a Pythium ophanidermatum, a Monilinia fructicola, a Glomerellaa cingulata, a Sclerotinia spp., a Sclerotinia sclerotiorum, or a Sclerotina homeocarpa.

3. The method of claim 1, wherein the composition is in the form of a liquid and each of the Bacillus licheniformis CH200 and the Bacillus subtilis CH201 are present at a concentration of from about 1.0×10⁸ CFU/ml to about 5.0×10¹³ CFU/ml.

4. The method of claim 3 wherein the composition comprises by weight percent:
0.5-40% of a biologically pure culture of not less than about 1×10¹¹ CFU/g Bacillus licheniformis CH200 deposited as DSM 17236, or a mutant thereof having all the identifying characteristics thereof;
0.5-40% of a biologically pure culture of not less than about 1×10¹¹ CFU/g Bacillus subtilis CH201 deposited as DSM 17231, or a mutant thereof having all the identifying characteristics thereof;
a liquid carrier;
a surface active agent; and
an adjuvant selected from the group consisting of viscosity modifiers, thickeners, preservatives, biocides or biostatic agents, antifreezes, crystallization inhibitors, suspending agents, dyes, anti-oxidants, foaming agents, light absorbers, mixing auxiliaries, antifoams, complexing agents, neutralizing or pH-modifying substances and buffers, corrosion inhibitors, fragrances, wetting agents, take-up enhancers, micronutrients, plasticizers, glidants, lubricants, and dispersants.

5. The method of any of claims 1, 2, 3 or 4 wherein the composition is a suspension concentrate comprising water and at least one surface active agent, and one or more additional adjuvants selected from thickeners, solvents, preservatives, antifreeze agents, and antifoam agents; optionally wherein the suspension concentrate is further diluted with water before delivery of the composition.

6. The method of any of claims 1, 2, 3, 4 or 5 wherein the liquid composition is a suspension concentrate comprising from 1 to 10 weight % of the Bacillus licheniformis culture or a mutant thereof having all the identifying characteristics thereof; from 1 to 10 weight % of the Bacillus subtilis culture or a mutant thereof having all the identifying characteristics thereof; 1 to 5 weight % of one or more surface active agent; and at least one thickener, solvent, preservative, antifreeze agent, or antifoam agent each independently comprising up to about 1 weight % of the composition.

7. The method of claim 1, wherein the composition is in the form of a dust, a dry wettable powder, a spreadable granule, or a dry wettable granule and each of the Bacillus licheniformis CH200 and the Bacillus subtilis CH201 are present in an amount of from about 1.0×10⁸ CFU/g to about 5.0×10¹³ CFU/g.

8. The method of claim 7, wherein the composition comprises a solid carrier selected from the group consisting of mono- or di-saccharides, oligo- or poly-saccharides, talc, titanium dioxide, pyrophyllite clay, attapulgite day, kleselguhr, silica, limestone, bentonite, calcium montmorillonite, sodium, potassium, magnesium, calcium or ammonium salts of acetate, carbonate, chloride, citrate, phosphate, or sulfate, cottonseed husks, wheat flour, soybean flour, pumice, wood flour, ground nut shells, lignin, yeast extracts, fish meal, or mixtures thereof.

9. The method of claim 8 wherein the composition comprises
5-40% of a biologically pure culture of not less than about 1×10¹¹ CFU/g Bacillus licheniformis CH200 deposited as DSM 17236, or a mutant thereof having all the identifying characteristics thereof;
5-40% of a biologically pure culture of not less than about 1×10¹¹ CFU/g Bacillus subtilis CH201 deposited as DSM 17231, or a mutant thereof having all the identifying characteristics thereof;
and maltodextrin, silica, calcium carbonate, or any mixtures thereof.

10. The method of claim 9 wherein the composition comprises 5-15% of maltodextrin.

11. The method of claim 9 wherein the composition comprises
5-20% of the culture of Bacillus licheniformis CH200 deposited as DSM 17236, or a mutant thereof having all the identifying characteristics thereof;
5-20% of the culture of Bacillus subtilis CH201 deposited as DSM 17231, or a mutant thereof having all the identifying characteristics thereof;
5-15% of maltodextrin;
35-45% of calcium carbonate; and
5-15% of silica.

12. The method of claim 1, wherein the composition is in the form of an oil dispersion and each of the Bacillus licheniformis CH200 and the Bacillus subtilis CH201 are present at a concentration of from about 1.0×10⁸ CFU/ml to about 5.0×10¹³ CFU/ml.

13. The method of any of claims 1-12 wherein the Bacillus licheniformis CH200 and the Bacillus subtilis CH201 are present in the form of spores or vegetative cells.

14. The method of any of claims 1-13, wherein the composition further comprises one or a combination of a microbial, a biological, or a chemical insecticide, fungicide, nematicide, bacteriocide, herbicide, plant extract, plant growth regulator, or fertilizer present in an amount suitable to benefit plant growth and/or to confer protection against a pathogenic infection in the plant.

15. A method for controlling plant fungal pathogens, the method comprising:
delivering to seed of a plant, roots of a plant, or soil surrounding a plant a combination of:
a first composition comprising a biologically pure culture of a Bacillus licheniformis CH200 deposited as DSM 17236, or a mutant thereof having all the identifying characteristics thereof; and
a second composition comprising a biologically pure culture of a Bacillus subtilis CH201 deposited as DSM 17231, or a mutant thereof having all the identifying characteristics thereof,
wherein delivery of the combination improves plant yield in the presence of a plant fungal pathogen, improves plant resistance to a plant fungal pathogen, or reduces plant infection by a fungal pathogen, and combinations thereof.

16. The method of claim 14, wherein the first and second compositions are in the form of a liquid and each of the Bacillus licheniformis CH200 and the Bacillus subtilis CH201 are present at a concentration of from about 1.0×10⁸ CFU/ml to about 5.0×10 CFU/ml.

17. The method of claim 14, wherein the first and second compositions are in the form of a dust, a dry wettable powder, a spreadable granule, or a dry wettable granule and each of the Bacillus licheniformis CH200 and the Bacillus subtilis CH201 are present in an amount of from about 1.0×10⁸ CFU/g to about 5.0×10¹³ CFU/g.

18. The method of claim 14, wherein the first and second compositions are in the form of an oil dispersion and each of the Bacillus licheniformis CH200 and the Bacillus subtilis CH201 are present at a concentration of from about 1.0×10⁸ CFU/ml to about 5.0×10¹³ CFU/ml.

19. The method of any of claims 14-18, wherein one or both of the first and second compositions further comprises one or a combination of a microbial, a biological, or a chemical insecticide, fungicide, nematicide, bacteriocide, herbicide, plant extract, plant growth regulator, or fertilizer present in an amount suitable to benefit plant growth and/or to confer protection against a pathogenic infection in the plant.

20. The method of claim 14 or claim 19, wherein the chemical fungicide is one or a combination of mefenoxam, fluopyram, chlorothalonil, thiophanate-methyl, fludioxonil, metalaxyl, or sedaxane.

21. The method of claim 14 or claim 19, wherein the chemical insecticide is one or a combination of thiamethoxam, pyrethroids, bifenthrin, tefluthrin, zeta-cypermethrin, organophosphates, chlorethoxyphos, chlorpyrifos, tebupirimphos, cyfluthrin, fiproles, fipronil, nicotinoids, or clothianidin.

22. The method of claim 14 or claim 19, wherein the chemical insecticide comprises chlorantraniliprole, cyantraniliprole, cyclaniliprole or indoxacarb, optionally in combination with imidacloprid, bifenthrin, thiamethoxam, pyrethroids, tefluthrin, zeta-cypermethrin, organophosphates, chlorethoxyphos, chlorpyrifos, tebupirimphos, cyfluthrin, fiproles, fipronil, nicotinoids, or clothianidin.

23. The method of any of claims 1-22, wherein the plant comprises corn, soybean, cotton or peanut.

24. The method of claim 23, wherein the plant comprises soybean or corn and wherein delivery of the composition improves plant yield in the presence of a plant fungal pathogen.

25. Use of a plant seed coated with a composition for controlling plant fungal pathogens, the composition comprising:
spores of a biologically pure culture of Bacillus licheniformis CH200 deposited as DSM 17236, or a mutant thereof having all the identifying characteristics thereof; and
spores of a biologically pure culture of Bacillus subtilis CH201 deposited as DSM 17231, or a mutant thereof having all the identifying characteristics thereof.

26. The use of claim 25, wherein the plant fungal pathogen comprises one or more of a Botrytis spp., a Botrytis cinerea, a Fusarium spp., a Fusarium colmorum, a Fusarium oxysporum, a Phytophthora spp., a Phytophthora capsici, a Rhizoctonia spp., a Rhizoctonia solani, a Magnaporthe oryzae, a Pythium spp., or a Pythium ophanidermatum.

27. The use of any of claims 25, 26 or 27 wherein each of the Bacillus licheniformis CH200 and the Bacillus subtilis CH201 are present in an amount ranging from about 1.0×10² CFU/seed to about 1.0×10⁹ CFU/seed.

28. The use of any of claims 25, 26, 27 or 28 wherein the plant seed comprises the seed of corn, soybean, cotton or peanut.

29. The use of claim 28, wherein the plant seed comprises soybean or corn.

30. The use of any of claims 25, 26, 27, 28 or 29, wherein the Bacillus licheniformis CH200 and the Bacillus subtilis CH201 are present in the form of spores or vegetative cells.

31. The use of any of claims 25, 26, 27, 28, 29 or 30, wherein the seed is further treated with one or a combination of a microbial, a biological, or a chemical insecticide, fungicide, nematicide, bacteriocide, herbicide, plant extract, plant growth regulator, or fertilizer present in an amount suitable to benefit plant growth and/or to confer protection against a pathogenic infection in the plant.

32. The use of claim 30, wherein the chemical fungicide is one or a combination of mefenoxam, fluopyram, chlorothalonil, thiophanate-methyl, fludioxonil, metalaxyl, or sedaxane.

33. The use of claim 30, wherein the chemical insecticide is one or a combination of thiamethoxam, pyrethroids, bifenthrin, tefluthrin, zeta-cypermethrin, organophosphates, chlorethoxyphos, chlorpyrifos, tebupirimphos, cyfluthrin, fiproles, fipronil, nicotinoids, or clothianidin.

34. The use of claim 30, wherein the chemical insecticide comprises chlorantraniliprole, cyantraniliprole, cyclaniliprole or indoxacarb, optionally in combination with imidacloprid, bifenthrin, thiamethoxam, pyrethroids, tefluthrin, zeta-cypermethrin, organophosphates, chlorethoxyphos, chlorpyrifos, tebupirimphos, cyfluthrin, fiproles, fipronil, nicotinoids, or clothianidin.

35. The use of claim 30, wherein the chemical insecticide and the chemical fungicide comprise a combination of fludioxonil, mefenoxam, thiophanate-Methyl, and thiamethoxam.

36. A composition comprising by weight percent:
0.5-40% of a biologically pure culture of not less than about 1×10¹¹ CFU/g Bacillus licheniformis CH200 deposited as DSM 17236, or a mutant thereof having all the identifying characteristics thereof;
0.5-40% of a biologically pure culture of not less than about 1×10¹¹ CFU/g Bacillus subtilis CH201 deposited as DSM 17231, or a mutant thereof having all the identifying characteristics thereof;
a liquid carrier;
a surface active agent; and
an adjuvant selected from the group consisting of surface active agents, viscosity modifiers, thickeners, preservatives, biocides or biostatic agents, antifreezes, crystallization inhibitors, suspending agents, dyes, anti-oxidants, foaming agents, light absorbers, mixing auxiliaries, antifoams, complexing agents, neutralizing or pH-modifying substances and buffers, corrosion inhibitors, fragrances, wetting agents, take-up enhancers, micronutrients, plasticizers, glidants, lubricants, and dispersants.

37. The composition of claim 36 wherein the composition is a suspension concentrate comprising water and at least one surface active agent, and one or more additional adjuvants selected from thickeners, solvents, preservatives, antifreeze agents, pH-modifiers and antifoam agents.

38. The composition of claim 37 comprising from 1 to 10 weight % of the Bacillus licheniformis culture or a mutant thereof having all the identifying characteristics thereof; from 1 to 10 weight % of the Bacillus subtilis culture or a mutant thereof having all the identifying characteristics thereof; 1 to 5 weight % of one or more surface active agent; at least one antifreeze agent; and at least one thickener, preservative, or antifoam agent each independently comprising up to about 1 weight % of the composition.
